(19) United States
(12) Patent Application Publication
(10) Pub. No.: US 2025/0028986 A1
(43) Pub. Date: Jan. 23, 2025

(54) ARTIFICIAL INTELLIGENCE FOR SOLVING DOMAIN-SPECIFIC PROBLEMS

(71) Applicant: Artificial Genius, Inc., Malibu, CA (US)

(72) Inventor: Paul Burchard, Jersey City, NJ (US)

(21) Appl. No.: 18/779,296

(22) Filed: Jul. 22, 2024

Related U.S. Application Data
(63) Continuation of application No. PCT/US24/38550, filed on Jul. 18, 2024.
(60) Provisional application No. 63/527,690, filed on Jul. 19, 2023.
(60) Provisional application No. 63/603,160, filed on Nov. 28, 2023.
(60) Provisional application No. 63/573,981, filed on Apr. 3, 2024.

(51) Int. Cl. G06N 5/045; G06N 3/00

(52) U.S. Cl. CPC G06N 5/045; G06N 3/002

(57) ABSTRACT

Aspects herein describe new methods of providing explainable solutions to domain-specific problems using artificial intelligence. A domain-specific problem, for example, a biological problem, is identified by a computational unit. The computational unit determines an initial feature subset comprising various features for observable characteristics of the problem. The computational unit optimizes the initial feature subset to improve the accuracy of the subset in generating a solution to the problem. The computational unit trains one or more predictive artificial intelligence models, based on the optimized subset, to output solutions to the problem. Subsequently, the computational unit utilizes the trained predictive model to output solutions to one or more additional domain-specific problems. The computational unit may additionally provide explainable solutions to the domain-specific problems by outputting a representation of steps performed by the predictive model to output the solutions.

### CROSS REFERENCE TO RELATED APPLICATIONS

This application claims priority to provisional U.S. Application Ser. No. 63/527,690, filed Jul. 19, 2023, and entitled “ARTIFICIAL INTELLIGENCE METHODS FOR SOLVING BIOLOGICAL PROBLEMS”; provisional U.S. Application Ser. No. 63/603,160, filed Nov. 28, 2023, and entitled “ARTIFICIAL INTELLIGENCE METHODS FOR SOLVING BIOLOGICAL PROBLEMS”; provisional U.S. Application Ser. No. 63/573,981, filed Apr. 3, 2024, and entitled “EXPLAINABLE ARTIFICIAL INTELLIGENCE METHODS FOR SOLVING BIOLOGICAL PROBLEMS”; and is a Continuation of Patent Cooperation Treaty Ser. No. PCT/US2024/38550, filed Jul. 18, 2024, and entitled “ARTIFICIAL INTELLIGENCE FOR SOLVING DOMAIN-SPECIFIC PROBLEMS,” each of which is hereby incorporated by reference in its entirety for all purposes.

### FIELD

Aspects described herein relate to computers, software, and artificial intelligence. More specifically, aspects relate to creation of explainable artificial intelligence models for solving domain-specific problems.

### BACKGROUND

The world of artificial intelligence and machine learning can be broadly divided into two opposing approaches: (1) the automated creation of generic artificial intelligence models that are intended to be used in any domain, and (2) the manual creation of domain-specific artificial intelligence models based on human expertise.

One drawback of conventional methods for automated creation of generic models is that they are not explainable, meaning that there is no mechanism by which a human expert may view, interpret, and/or otherwise explain the decisions, processes, steps, or the like used by the machine learning model to generate an output (e.g., a solution to a domain-specific problem, as described herein). Generic artificial intelligence models generated by automated methods attempt to learn complex, domain-specific patterns from simple, generic input features (e.g., unstructured observational information corresponding to a domain-specific problem, commonly known characteristics of one or more objects, environmental conditions, or the like corresponding to a domain-specific problem, and/or other features of a domain-specific problem). Such models conventionally comprise complex functional forms (e.g., a large language model, a deep neural network, or the like) which often require significant resources (time, money, or the like) to generate a training set of simple features large enough to train the model to a desired degree of accuracy. Further, the inner workings of such models are inscrutable. That is, the processes, decisions, correlations, and/or other factors used by the models to output a solution to a domain-specific problem are “black box,” meaning a human expert cannot determine the factors that lead to a specific solution. As a result of this lack of explainable inner workings these generic models cannot be validated, improved, or relied upon by human domain experts, and their only output is a final prediction which cannot be verified or used for any deeper domain analysis or reasoning. Another major drawback of these generic models is that, because their design has no specialized connection to the domain in which they are being applied, training them from the simple generic features described above requires enormous amounts of data. Obtaining the necessary domain-specific data may be too expensive to make such an approach viable, in at least some examples.

Conventional methods of manually creating domain-specific artificial intelligence models (e.g., based on human expertise) have many drawbacks as well. For example, the development of such models is costly in terms of the combination of both the domain expertise and artificial intelligence expertise that is required to construct them, one model at a time. Due to this manual, one-off process, such models cannot be repurposed, even for other problems in the same domain, due to the specificity of their design and construction. Conventional techniques for manual generation of such models requires significant amounts of time from domain experts be devoted to the construction and selection of explainable features that are suitable to solve the particular domain-specific problem.

Examples of the above-described deficiencies in conventional methods of solving domain-specific problems using artificial intelligence can readily be seen in, for example, the domain of biology. Automated creation of generic artificial intelligence models having forms such as, for example, deep neural networks or transformer models have been applied to biological problems, but with mixed results and a lack of explainability. For example, one such generic artificial intelligence model, the AlphaFold protein folding algorithm, is based on training deep neural networks on a very large number of known protein configurations. Due to the enormous data requirements of this type of generic model, its training was only made possible due to significant financial investment on the order of billions of dollars having previously been spent on experimentally determining these known protein configurations, and the public availability of this data. Further, the AlphaFold model does not produce a solution to the protein folding problem that can be replicated—it merely provides a non-explainable prediction of a final folded configuration of an amino acid sequence. Although the prediction of this configuration by AlphaFold may have a desired degree of accuracy, the model does not output any form of explanation as to how the resulting protein was folded into that configuration. Due to this lack of explainability, there is no way to validate the output other than to perform the experiment whose cost the artificial intelligence model was designed to avoid. Biological proteins are only evolutionarily viable if they fold properly into the correct, biologically active configuration, via an incremental and dynamic process that takes place during their biological synthesis. Conventional models, such as AlphaFold, do not produce an explanation of the dynamic process required to produce the prediction outputted by the model.

Conventional techniques for manual generation of artificial intelligence models for solving domain-specific problems in biology are similarly deficient. For example, some conventional methods for generating artificial intelligence models for solving biology problems involve the construction of “molecular fingerprints” or “reaction fingerprints”. These fingerprints are vectors of molecular features (e.g., molecular substructures in molecules involved in a biological process, reactants, or the like), manually created by domain experts in, for example, biochemistry, that attempt to identify substructures in molecules that could be relevant to solving a biological problem. Because the relevancy of a given molecule or molecular substructure is dependent on the biological problem being solved, there are numerous, competing, manually-defined standards for such fingerprints. To solve any particular biological problem thus require the manual effort of domain experts to be repeated for each such problem, in order to determine the fingerprints that are relevant to solving the problem. This can lead to significant costs in terms of time, money, and/or other resources required to solve a biological problem using manual generation of artificial intelligence models for solving domain-specific problems.

As such, there exists a strong need for methods of generating and using artificial intelligence models to solve domain-specific problems that overcome the deficiencies of both conventional approaches described herein. To overcome the deficiencies associated with automated generation of artificial intelligence models based on simple features, there exists a need for methods of generating artificial intelligence models based on features corresponding to an optimal level of detail for the given domain and domain-specific problem being solved. To further appreciate the need for such methods, consider again the example of the domain of biology.

Biology is an extremely complex subject due, in part, to the numerous levels of description that are already available to study it. For example, one can look at the ecological, taxonomic, genomic, proteomic, molecular, atomic physics levels, and/or other levels. Of these, the highest level descriptions tend to have the most structure to exploit for prediction, but these levels are the least directly connected to the actual complexities of the problem, which must be resolved by quantum physics. On the other hand, starting at the lowest levels, with the actual quantum physics, leads to intractable calculations, and misses the structure and restrictions imposed by the higher level descriptions which could simplify the problem. For example, chemical reactions depend on the binding energies of the various atoms involved in the reaction. That sounds like a quantum physics problem, but those binding energies are not a constant of the two coupled atoms; they are dependent on the surrounding molecular structure. As a result, the atomic physics level, despite being the ultimate arbiter of outcomes, is too low a level to be useful for computationally feasible and accurate prediction.

Given this plethora of existing descriptive levels for biology, conventional methods have yet to identify the optimal level of description for the purposes of training a predictive artificial intelligence model. Accordingly, there exists a strong need for criteria method of training an artificial intelligence model to solve domain-specific (e.g., biological) problems at an optimal level of description of the problem and using features corresponding to the optimal level of description. There further exists a need to provide explainable, white-box predictions using such a model that can be understood, verified, and used for purposes beyond mere prediction in order to harness the results of training a predictive model at the optimal level of description.

### SUMMARY

The following presents a simplified summary of various aspects described herein. This summary is not an extensive overview, and is not intended to identify key or critical elements or to delineate the scope of the claims. The following summary merely presents some concepts in a simplified form as an introductory prelude to the more detailed description provided below.

In order to address the above deficiencies in conventional methods of solving domain-specific problems using artificial intelligence (AI), and provide additional benefits that will be realized upon reading the disclosure, elements of the illustrative aspects described herein address new improvements to applying artificial intelligence methods to solving domain-specific problems.

Methods for solving domain-specific problems, as described herein, may comprise identifying, for a domain, an optimal level of description for the domain. For example, a human domain expert analyses, observational information, and/or other data of the domain may be used in a one-time process to define an optimal level of description for AI models to consider when solving problems corresponding to the domain. This optimal level of description may be used to produce candidate features (e.g., by a computer system and/or a predictive AI model) relevant to solving a domain-specific problem. The optimal level of description may comprise descriptive information that indicates distinctions between candidate features corresponding to potential predictive values. A size of the descriptive information may be below a threshold storage capacity. The descriptive information may comprise all information identified (e.g., by a predictive AI model, a human expert, and/or other sources) as necessary to predict a solution to the domain-specific problem. These methods as applied to, for example, the domain of biology, as described herein, may comprise reducing the problem from an ecological, taxonomic, genomic, proteomic, molecular, quantum physics, or other standard descriptive level, to the previously undefined descriptive level of molecular mechanisms of interaction, from which data corresponding to the logical operation or non-operation of biological pathways may be constructed. These molecular mechanisms may be generated, by a predictive AI model trained at the optimal level of description, by means of combinatorial algorithms that combine features or sub-features of the domain-specific problem at the optimal level of description.

Methods for providing AI models that produce explainable solutions, as described herein may include one or more of (1) generating domain-specific candidate features at the optimal level of description for the domain (e.g., via a combinatorial algorithm, or the like); (2) selecting the specific candidate features that are involved, either positively or negatively, in affecting the outcomes of the domain-specific problem being solved; (3) eliminating redundancy between similar candidate features with similar predictive effects on the outcomes of the domain-specific problem; (4) compressing similar candidate features with similar effects on the outcomes of the domain-specific problem, into compound features that are explainable and correspond to the desired level of description of the domain; (5) collecting the resulting selected, non-redundant, and/or compressed candidate features as an optimized feature subset of explainable features for a predictive model; and/or (6) training of a predictive model based on the optimized feature subset, using simple functional forms that produce explainable solutions to domain-specific problems

The present disclosure provides multiple improvements over conventional AI applications to solving domain-specific problems. For example, as described herein, reducing domain-specific problems to the optimal level of description and identifying optimal candidate features to incorporate into a feature set improves the scope and level of detail in auxiliary data gathered while applying AI models to solve domain-specific problems. Using the optimal level of description includes more complex interactions than those used in automated methods to train conventional AI models. Further, the use of simplified forms of AI model (e.g., shallow neural networks, or the like) provides transparency in the steps AI models trained using the methods described herein take to produce solutions to domain-specific problems. This transparency allows deeper insights and extrapolation from the process of solving a domain-specific problem than conventional methods of manually generating AI models to solve domain-specific problems.

The various aspects of the illustrative embodiments are substantially shown in and/or described in connection with at least one of the following figures, as set forth more completely in the claims.

These and other advantages, aspects, and novel features of the present disclosure, as well as details of illustrated embodiments, thereof, will be more fully understood from the following description and drawings.

### BRIEF DESCRIPTION OF THE DRAWINGS

A more complete understanding of the present invention and the advantages thereof may be acquired by referring to the following description in consideration of the accompanying drawings, in which like reference numbers indicate like features, and wherein:

FIG. 1 depicts an illustrative network environment that may be utilized in accordance with various embodiments;

FIG. 2 illustrates an example user device that may be used in a network environment, such as either a client device or a server in the network environment of FIG. 1, in accordance with illustrative aspects described herein;

FIG. 3 illustrates a general view of interactions between a user device and a computational device during iterations of the method according to illustrative aspects described herein;

FIG. 4 illustrates steps of providing artificial intelligence for solving domain-specific problems according to illustrative aspects described herein; and

FIG. 5 illustrates steps of optimizing feature sets according to illustrative aspects described herein.

### DETAILED DESCRIPTION

### I. Description of Foundational Algorithms

### A. Method for the Automated Generation of Explainable, Domain-Specific AI Models

To address the problems with applying conventional artificial intelligence to domain-specific problems, it is important to identify the optimal level of description of the domain-specific problem. A middle ground is needed between the highest level descriptions that have a lot of structure but do not include sufficient detail to make good predictions, and the lowest level descriptions that are so detailed and structureless that they are computationally infeasible. The optimal level of detail may be identified based on balancing an estimated (e.g., by a human expert, machine, artificial intelligence, or the like) minimum of information required to solve a domain-specific problem and an estimated amount of computing resources (e.g., computation time, memory, or the like) required to solve the domain-specific problem. In this way, the optimal level of detail may be identified such that the optimal level of detail corresponds to descriptive information, corresponding to the domain-specific problem, that indicates distinctions between candidate features corresponding to potential predictive values. A size of the descriptive information may be below a threshold storage capacity, in order to preserve computing resources. In some examples, the descriptive information may comprise all of the information identified as necessary (e.g., by a predetermined set of rules for solving domain-specific problems, by a human expert, and/or by other means) for solving the domain-specific problem.

The present disclosure provides an illustrative example of applying the disclosed methods of automated generation of explainable, domain-specific AI models to the domain of biology. It should be understood that these methods are applicable to any domain and that the biology examples are non-limiting. As described herein, with respect to biological problems, the molecular level of provides approximately the optimal balance between high-level structure and low-level detail. However, conventional methods that utilize the molecular level of description do not account for the concepts that (1) what matters is not individual molecules, but the interactions between multiple molecules that constitute steps in the overall biochemical pathways of interaction through which biology operates; (2) the relevant information about any of the individual molecules involved in any such an interaction in the biochemical pathways cannot be captured by any fixed notion (e.g., conventional “fingerprinting” methods), but must be determined specifically for each such interaction step. To improve over the conventional methods, the present disclosure identifies the optimal level of analysis, for an optimal combination of feasibility and accuracy for predictive modeling in biology, as the level of molecular mechanisms. These molecular mechanisms may comprise the minimal information about any one step in the biochemical pathways that is necessary to determine its logical operation or non-operation, and in the cases that it is operational, what the resulting outputs would be that could be either desirable or undesirable inputs for one or more next steps in the pathways. For example, a given molecular mechanism may comprise molecular substructures involved in the step of a biochemical pathway, geometric constraints between one or more molecular substructures corresponding to a single molecule (e.g., of a plurality of molecules) involved in the step of the biochemical pathway, catalysts affecting a rate of the step of the biochemical pathway, environmental conditions affecting the step of the biochemical pathway, and/or other mechanisms.

By this definition, a molecular mechanism may include combinations of molecular substructures in one or more molecules involved in the interaction, which jointly and directly contribute to a positive or negative effect on the outcome or rate of a potential molecular interaction, such as reaction or binding, or a downstream effect of such an interaction. These substructures may be unique to each such interaction. A molecular mechanism may further include geometric relationships between multiple substructures within a single molecule, if such a combination of substructures is required to contribute to a positive or negative effect on the outcome or rate. A molecular mechanism may include any cofactors or catalysts that contribute to a positive or negative effect on the outcome or rate. A molecular mechanism may further include environmental information (e.g., temperature, pH level, or the like), that is required for the interaction to occur. The substructures should be large enough to constrain the energies of the critical atoms and bonds directly involved in the interaction, but small enough to contain no more than the necessary number of atoms and bonds to provide sufficient accuracy. Thus, the first step of the present method is to break down the original biological problem into one or more sub-tasks of automatically finding and quantifying the relevant molecular mechanisms, and creating predictive models based on those mechanisms. For example, if the original problem is expressed in terms of amino acid sequences, a given amino acid sequence can be translated into an actual protein or polypeptide molecule, and then specific molecular mechanisms for interaction of that protein or polypeptide molecule with other relevant molecules may be automatically extracted.

The present method allows for some flexibility in the construction of molecular mechanisms, which may include overdetermined and/or underdetermined specifications of various aspects of the complete molecular mechanism. For example, overdetermination may be useful in order to provide better ability for the predictive model to generalize beyond the available, potentially limited, training data, while underdetermination may be useful in order to adapt to any constraints in the available training data that prevent determination of the complete mechanism. Some examples of this flexibility will now be presented to show the range of possibilities, which can be expanded to other cases by those skilled in the art.

One example where overdetermination may be useful is in the substitution of similar atoms (e.g., in the same column of the periodic table) within molecular substructures involved in the interaction. Instead of creating a large number of similar features which implement those substitutions, which may each have similar effects on the outcome and reduce the ability of the predictive model to generalize well beyond the training data, the feature specification may include molecular substructures that have multiple choices for particular atoms. Another example where overdetermination may be useful is in the specification of cofactors or catalysts. In some cases, a variety of cofactors or catalysts may have similar effects on a positive or negative outcome of the interaction. In such cases, analogously to the atom substitution, the feature specification can include multiple choices for the cofactors or catalysts, and potentially quantitative information on the number of such required to affect the interaction either positively or negatively.

One example where underdetermination may be useful is when multiple substructures of the same molecule are involved, positively or negatively, in affecting the relevant molecular interactions, as can happen, for example, with interactions between larger molecules. In such cases, the geometric relationships of these multiple substructures may be important to the outcome or rate. Although such geometric constraints can be included into the feature specification itself, in order to make a fully determined specification of the complete molecular mechanism, another alternative is to include the geometric information as additional quantitative features in the predictive model, with the main features representing underdetermined molecular mechanisms that only include at most one of the multiple substructure from each molecule involved in the potential interaction in each feature. This can be useful if the simple functional form of the predictive model is likely to make better use of the geometric information than the construction of features based on completely determined molecular mechanisms.

Another example where underdetermination may be useful is when the training data only includes information on one side of the interaction. In such cases, original training data may be augmented with a previously constructed database of molecular mechanisms (as explained below) that enables the identification of the other side of each interaction. For example, in analyzing human diagnostic data that only includes information on biomarkers, which when translated into molecular terms constitute only one side of an interaction by which the biomarker affects human biology, the training data can be augmented with a database of known molecular mechanisms in human biological pathways, for which the biomarker could provide one side of the mechanism, and thereby engage with known biological pathways. If such a database of molecular mechanisms is not available, or if there is a possibility that the other side of the molecular mechanism is not yet known and its discovery could be part of the biological problem to be solved, then underdetermination techniques may be applied by using features that include only one side of the molecular mechanism. In such cases, the present method can still provide deep insight through the construction of underdetermined features that only include one side of the interaction, thereby providing explicit clues for further investigation. To recap the example of analyzing human diagnostic data, if the relevant disease pathways in the human are not yet understood, the present method can automatically identify potentially active sites on the molecules in the diagnostic assay, providing clues for the identification of as-yet-unknown molecules in the disease pathway.

Despite the flexibility in specification of features described herein, it is important to note again the distinction between all of the variations of the present method, and existing methods based on fingerprints as described herein, which also involve molecular substructures. First, as previously explained, existing fingerprints are not identified automatically by artificial intelligence, but rather by expert human input. This results in either generic fingerprints that may not have relevance to a particular biological problem, or highly specialized fingerprints constructed for a specific problem through painstaking experimental work. In contrast, the present method identifies the most relevant molecular substructures, customized for every possible biological problem, completely automatically from the relevant data via artificial intelligence. Second, Conventional fingerprinting methods also do not include as features complete molecular mechanisms constituting the fundamental irreducible elements of the pathways by which biology operates. Instead, the features in conventional fingerprints may only include substructures from one molecule at a time, relying on the complex functional forms of the predictive model built on such features to automatically make the connections between the substructures of the various molecules involved in the complete mechanism. This reliance on complex functional forms causes inaccuracies when attempting to identify the specific and complex nature of the biological patterns present in the data provided as input to the models. In contrast, the present method considers complete molecular mechanisms as a fundamental unit of feature construction.

The first step of identifying relevant molecular mechanisms may comprise acquiring, either through existing data sets, or through laboratory experiments, sufficient data to evaluate which combinations of substructures and their geometric relationships of the one or more molecules involved in relevant interactions, and which cofactors or catalysts, are reliably disposed to either excite or inhibit those interactions, or differentially affect their downstream effects, across a sufficiently large and diverse subset of the data, and thereby constitute useful and meaningful features for the predictive model. The mechanisms so identified need not be perfectly predictive in their own right, only to have a sufficiently large and reliable effect on the outcome, rate, or downstream effect, so that the subsequent predictive model built in the second step on a feature set that includes those mechanisms may make the desired accurate predictions. This identification can be performed completely automatically, and made computationally feasible by placing reasonable limits on the potential substructures and geometries considered. For example, if one of the molecules is a protein, in some examples the protein backbone may not be relevant to the outcome, because the backbone is identical along the length of that entire protein, or any other protein; as a result, the large number of potential substructures that include the protein backbone may be ignored. As another example, each individual relevant substructure of a molecule participating in the interaction may have both lower and upper limits on its size, based on practical considerations about the size of a substructure necessary to determine the energies of the atoms and bonds that directly participate in the interaction.

In the case where the available data includes a database of known molecular interactions, such as reactions or non-covalent bindings, the method may identify the exact atoms and bonds that are directly involved in the interaction. For example, in analyzing a known chemical reaction, preserved substructures between the reagents and the products may be automatically identified by molecular matching, in order to first identify, by elimination, the specific bonds that were not preserved, and instead reorganized, during the reaction. Then, second, a molecular mechanism for the reaction may be constructed as a combination of suitably sized molecular substructures surrounding each of the changed bonds in the reagents.

In cases where the training data for a predictive model only includes a set of molecules (e.g., after translation of any biomarkers on different levels of biological description into molecular terms), and experimental results about the effects of these molecules, the method operates by automatically screening all possible one sided features of the nature described above, within the above described constraints, and evaluating them for inclusion in the model feature set, based on a variety of criteria which may include: (1) largest differential effects on the outcomes, rates, or downstream effects of the interactions, (2) compatibility with any known information about the nature of the interactions involved, and (3) compatibility with any known auxiliary data on complete molecular mechanisms for relevant interactions in which the possible feature could participate. If auxiliary data is applied, then the resulting features will be complete molecular mechanisms; otherwise, they will be one sided, requiring further investigation.

Using the novel methods of identifying an optimal level of description for a domain (e.g., molecular mechanisms, for the biology domain) as described herein, a novel method for the automated generation of explainable, domain-specific artificial intelligence models for solving domain-specific problems may be achieved. For example, a predictive AI model may be generated, based on using molecular mechanisms as the explainable model features, for solving biological problems.

The present method offers advantages over traditional methods: (1) due to the complex but meaningful and domain-specific nature of the features provided to predictive models generated as described herein, and the simplicity of the functional forms taken by these predictive models, the methods described herein produce “white-box” models that go beyond mere prediction, to provide deep and verifiable insights on how to connect the results to other parts of the overall network of biological pathways, or even to re-engineer the biology to produce better outcomes; (2) because deep, domain-specific knowledge about biology is embedded in the construction of the features themselves, the present method provides improvements in efficiency over traditional methods, which are based on the hope or assumption that biologically relevant patterns may emerge from attempting to fit generic complex functional forms that have no special understanding of biology. For example, a diagnostic test for a disease based on the methods described herein would provide more than just an opaque, unverifiable yes/no answer; it would further provide connections to biological pathways involved in the disease that resulted in the yes/no diagnosis, and thereby clues for the development of potential therapeutic interventions.

Generating a predictive model may comprise generation of candidate features (e.g., via a combinatorial algorithm), specific to each domain-specific problem to be solved. For example, candidate molecular mechanisms for a specific biological problem may be performed as described herein. This combinatorial generation may result in a large number of candidate features. Generating the predictive model may further comprise the selection of candidate features based on their predictive value for the specific biological problem, as described herein.

Generating the predictive model may comprise performing automatic feature redundancy elimination. Depending on the size and nature of the training data, the screening method described above may produce multiple similar features with similar differential effects. For example, molecular substructures that differ by one atom may not be distinguishable in their effects based on available data. Redundant features reduce the ability of the resulting predictive model to generalize beyond the training data, so the redundancy elimination should be chosen to best avoid the kinds of generalization errors that can be anticipated for the specific biology problem being solved. In many cases, the concern would be false positives, resulting either from selecting features that are too small and don't include enough structure to be determinative on a broader set of data, or from selecting features that are too large to be causative in their own right, but happen to be statistically correlated with outcomes. In such cases, if there are two similar features with similar differential effects, the one including a larger number of atoms, but still within reasonable size constraints that could contribute to determination of the energies of the relatively small number of atoms or bonds that actually participate in the interaction, would be chosen and the other discarded, as it is likely to generate fewer false positives. Additional automatic redundancy elimination criteria may be added by those skilled in the art, depending on the nature of the specific biological problem.

Generating the predictive model may comprise compression of similar features with similar predictive effects into a single complex feature at the optimal level of description. Such similar features might otherwise present statistical confounding effects for any predictive model built on them. With regard to molecular mechanisms, one method of compression may be to combine biochemically similar molecular mechanisms into a single, more complex specification of a molecular mechanism that allows some variation in its specification. For example, two molecular mechanisms that are identical, except for the substitution of an atom by another atom in the same column of the periodic table, can be combined into a single molecular mechanism that has multiple choices at that atomic location. As another example, two molecular mechanisms that are identical, except for the geometric constraints between multiple substructures, can be combined into a single molecular mechanism that has a range of allowed geometric constraints between those substructures.

As mentioned, the above design of the feature sets incorporates domain specific knowledge about biology that improves the accuracy of predictions. Based on these features, relatively simple predictive models can be constructed. There is, however, one important characteristic preferred for the chosen predictive modeling method, which is that it be able to make predictions with sharp transitions or even discontinuities in the underlying features. This is fundamental to the nature of the biological problem. For example, the binding of a reasonably sized small molecule to an enzyme may require multiple binding sites to be successful, and the dependence of successful binding on the geometric relationship between those binding sites on the enzyme may vary, transitioning from highly excitatory to highly inhibitory as the distance between the same binding sites on the enzyme is changed.

Examples of predictive modeling methods that meet this criterion include shallow neural networks, whose final prediction layer has activation functions with sharp transitions in either the zeroth or first derivative. For example, final sigmoid activations can be used for classification tasks, or final rectified linear activation functions (relu activations) can be used for numerical prediction tasks. There is typically no need for deep neural networks, as most of the work has been done by the feature set. Another example of a predictive modeling method that meets this criterion is decision trees for classification tasks, or its analog, regression trees for numerical prediction tasks. Another example, for classification tasks, would be ranking functions on the resulting classes. Ranking functions can be constructed by predicting class rank difference from individual features, and then finding the best-fit overall ranks by aggregating these individual rank differences.

Solving biological problems by breaking them up into sub-tasks that can be solved by the present AI methods may take many forms, which will be apparent to those skilled in the art. To take one example, to develop therapeutic interventions for a disease, whose biological pathways are not known, one can begin with the sub-task of producing a predictive diagnostic test based on patient assays. By acquiring a limited amount of training data on such assays and verified diagnoses, the present AI methods may produce a predictive diagnostic test based on explainable features which are underdetermined molecular mechanisms providing the automatically determined substructures of the molecules involved in the assay which connect to the unknown disease pathway. Then, as the next sub-task, additional training data comprising known human metabolic pathways may be used to determine which pathways are actually activated by interactions involving the molecular substructures identified in the first sub-task. As a final sub-task, the human metabolic pathways identified by the previous sub-task may be evaluated for intervention by drug candidates.

### B. Illustrative Application of Artificial Intelligence for Solving Domain-Specific Problems

In the following description of various embodiments, illustrative steps to implement the above AI methods will be detailed. It should be understood that one or more additional or alternative steps may be performed using methods described herein without departing from the scope of this disclosure. Although the steps described herein are generally discussed in relation to solving a biological problem, it should be understood that this description is merely illustrative and that the methods described herein may be performed to apply artificial intelligence to solving other domain-specific problems without departing from the scope of this disclosure. We will take as an example the problem of learning a diagnostic model from a biomarker assay performed on a panel of human patients who have been assigned various diagnostic categories by expert physicians. The categories could be as simple as yes/no, the patient has the disease, or more precise and fine-grained, providing distinct diagnostic categories for different stages, variants, or physiological expressions of the disease. A further objective of applying the AI methods described herein may be to use this assay data to go beyond mere black-box diagnostic prediction, to provide fine-grained, causal explanations for each diagnostic prediction, based on the human biological pathways found to be activated in each patient.

The biomarker assay dataset may comprise a table, which for each combination of biomarker and patient case ID, provides two pieces of data: (1) some measure of the amount of the biomarker present in the patient's case, as measured by means of some biochemical assay, such as, for example, various types of genomic sequencing, chromatography, or electrophoresis; and (2) the diagnostic category assigned to that patient case.

For the purposes of applying the present AI methods, a computer system, computational unit, or the like may translate the biomarker information from its original level of biological description into the molecular level, from which the method will subsequently and automatically extract molecular mechanisms. For example, DNA or RNA sequences may be converted either into DNA or RNA molecules, if the molecules themselves are directly involved in the relevant interactions, or into their translation products, i.e., amino acid sequences, which can themselves be further converted into peptide molecules such as proteins and enzymes. In preferred embodiments, the final quantitative information for each resulting molecule in the assay would be converted into some type of molar units. Thus, the inputs of the translated assay dataset will be taken to be assay molecules and patient IDs, and for each such pair, the outputs will be the molar concentration of the assay molecule for that patient, and the patient's diagnostic category.

In order to provide the benefit of identifying complete molecular mechanisms, assay dataset may be augmented with a database of known human biological pathways, including biochemical reactions and/or binding interactions, along with any enzymes or catalysts necessary for the interaction to proceed at a biologically acceptable rate. Each step in the pathway may have metadata that contains information about its biological function in the human body.

In order to obtain the molecular mechanisms corresponding to those pathways, each biochemical reaction may be analyzed in order to determine exact bonds that are rearranged to cause the reaction. This may be determined automatically by eliminating identical molecular substructures in the reagents and products of the reaction, leaving over only the small number atoms and bonds where the rearrangements took place. Then, the corresponding molecular mechanism may be taken to be the combination of correctly-sized neighborhoods of the changed atoms and bonds in the reagents and products, along with the required enzymes or catalysts. The size of the neighborhoods may be chosen to be large enough to sufficiently accurately determine the energies of the changed bonds, but small enough to not avoid including extraneous atoms and bonds that are not necessary for sufficiently accurate determination. Thus, the original database of human biological pathways may be converted into a database of molecular mechanisms that provide the information to determine the operation of each step of the pathways in the original database, along with any metadata on the specific biological function being performed by that particular step.

A large number of candidate molecular mechanisms may be generated from the assay data. This step starts out by generating random combinations of molecular substructures, or even all possible combinations of molecular substructures, of the assay molecules, with each molecular substructure of a reasonable size, and the geometric relationships between potentially multiple molecular substructures reasonably constrained, and selecting from combinations of molecular substructures the combinations that are sufficiently good matches to one side of a molecular mechanism present in the database of human pathways, whose metadata indicates that it might be relevant to the diagnostic problem being solved. For example, if the disease being analyzed is cancer, the matching molecular mechanisms from the database may be constrained to only include known functions related to the progression of cancer. The final candidate molecular mechanisms may comprise a first side comprising combinations of molecular substructures and geometric constraints in the assay molecules, sufficiently matching one side of suitable molecular mechanisms from the human pathways database, and a second side given by the other side of the molecular mechanism operating in the human, from the human pathways database.

Candidate molecular mechanisms may be selected from the above set. For example, candidate molecular mechanisms which have sufficiently differential effect on the diagnostic categories, as a function of the amount of the side of the molecular mechanism contained in the assay molecules. The amount of any candidate molecular mechanism may be measured in atomic molar units, by multiplying the molar amounts of the assay molecules by the number of atoms of the mechanism present in each, and adding the resulting amounts over all assay molecules that include the one side of the mechanism. If the number of diagnostic categories is greater than two (yes/no), then it may be useful to select candidate mechanisms that only have a sufficient differential effect on one pair of diagnostic categories.

The next step may be to perform redundancy elimination. The previous step may generate a large number of similar candidate mechanisms, for example, differing by a single atom, which have similar differential diagnostic effects, and would therefore act as confounding variables in any predictive model built on such features. The strategy for eliminating such redundancy, and choosing a single representative mechanism among many similar ones, is to designate a smaller mechanism as redundant, if for sufficiently many relevant patient cases, where the amount of the mechanism is sufficiently large, a sufficiently large fraction of the total amount of the smaller mechanism is contained within instances of some larger candidate mechanism that has also been selected in the previous step, has similar differential diagnostic effects, and is linked to the same complete mechanism in the human pathways database.

Feature compression may be performed, in order to further reduce potentially confounding variables. Multiple types of feature compression may be performed. Feature compression may involve combining similar candidate features (e.g., candidate molecular mechanisms) to prevent confounding variables (e.g., variables that might, due to their similarity in construction and/or predictive effects, cause a predictive AI model trained based on the variables to confuse their respective effects). In one example related to the biology domain, if two mechanisms are identical except for the substitution of similar atoms (for example, in the same column of the periodic table), then they may be combined into a single feature which has multiple options for the atom at that position. This may result in implicit generalization, if multiple such atoms occur in one mechanism, as all combinations of substitute atoms might not have occurred in the dataset. In an additional or alternative example, if two mechanisms are identical, except for the geometric constraints between multiple molecular substructures, then they may be combined into a single feature in which the geometric constraints take the form of a range of distances, instead of a definite distance. This should only be done if such ranges would not thereby implicitly include candidate features that have already been otherwise rejected (for example, as not having sufficient differential effects).

Feature scoring may be performed. For example, each feature in the feature set may be scored and ranked according to its predictive power, either positive or negative. In some examples, the feature score may also or alternatively be based on performing feature compression, performing redundancy elimination, and/or performing other optimization actions described herein. To produce the final feature set for the predictive model, an absolute or threshold score cutoff may be applied, below which features are rejected.

Based on constructing a set of features as described herein, an explainable, predictive, diagnostic model may be trained. For example, the model may be trained based on the biologically meaningful feature set constructed by the previous steps. The model itself may be simple and transparent. Some choices include decision trees, random forests, shallow neural networks, or ranking functions.

The predictive model may be applied to a biological problem to produce explainable diagnostic results. For example, given biomarker assay results for a new set of patients, the predictive model is applied, and in addition to the predicted diagnosis, the model can be inspected to see which biologically meaningful features most contributed to the result. By construction, each feature may be connected to a specific biological function within the complex of human pathways.

### II. Illustrative System Architecture

In the following description of the various embodiments, reference is made to the accompanying drawings, which form a part hereof, and in which is shown by way of illustration various embodiments in which the aspects described herein may be practiced. It is to be understood that other embodiments may be utilized and structural and functional modifications may be made without departing from the scope described herein. Aspects are capable of other embodiments and of being practiced or being carried out in various ways. Also, it is to be understood that the phraseology and terminology used herein are for the purpose of description and should not be regarded as limiting. Rather, the phrases and terms used herein are to be given their broadest interpretation and meaning. The use of “including” and “comprising” and variations thereof is meant to encompass the items listed thereafter and equivalents thereof as well as additional items and equivalents thereof. The use of the terms “mounted,” “connected,” “coupled,” “positioned,” “engaged” and similar terms, is meant to include both direct and indirect mounting, connecting, coupling, positioning and engaging. Although the various illustrative embodiments described herein generally provide examples of using this system architecture to apply predictive AI models to biological problems, it should be understood that this architecture may be used to generate and/or apply predictive AI models for solving any domain-specific problems, as described herein, without departing from the scope of this disclosure.

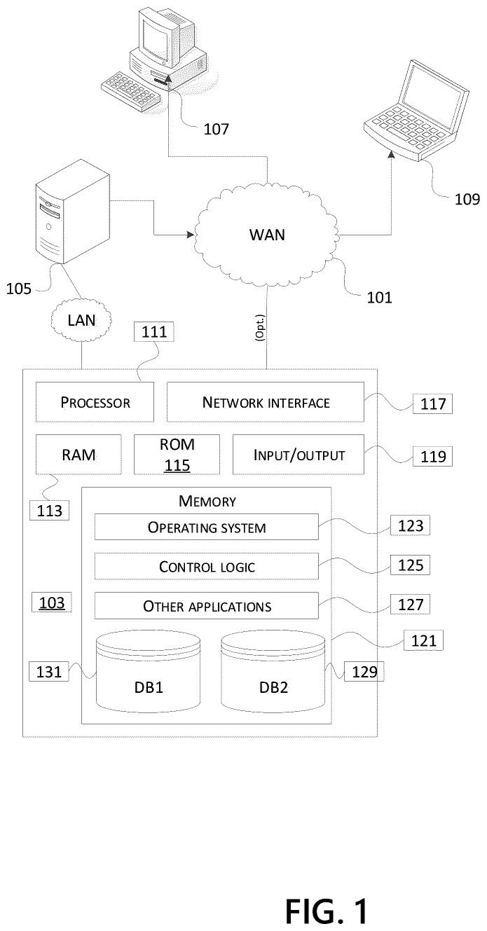

FIG. 1 illustrates one example of a network architecture and data processing device that may be used to implement one or more illustrative aspects. Various network nodes 103, 105, 107, and 109 may be interconnected via a wide area network (WAN) 101, such as the Internet. Other networks may also or alternatively be used, including private intranets, corporate networks, LANs, wireless networks, personal networks (PAN), and the like. Network 101 is for illustration purposes and may be replaced with fewer or additional computer networks. A local area network (LAN) may have one or more of any known LAN topology and may use one or more of a variety of different protocols, such as Ethernet. Devices 103, 105, 107, 109 and other devices (not shown) may be connected to one or more of the networks via twisted pair wires, coaxial cable, fiber optics, radio waves or other communication media.

The term “network” as used herein and depicted in the drawings refers not only to systems in which remote storage devices are coupled together via one or more communication paths, but also to stand-alone devices that may be coupled, from time to time, to such systems that have storage capability. Consequently, the term “network” includes not only a “physical network” but also a “content network,” which is comprised of the data—attributable to a single entity—which resides across all physical networks.

The components may include computational unit 103, web server 105, and client devices 107, 109. Computational unit 103 may be a general or special-purpose computer or computer farm. Computational unit 103 may be a computational unit that provides overall access, control and administration of databases and control software for performing one or more illustrative aspects described herein. Computational unit 103 may be connected to web server 105 through which users interact with and obtain data as requested. Alternatively, computational unit 103 may act as a web server itself and be directly connected to the Internet. Computational unit 103 may be connected to web server 105 through the network 101 (e.g., the Internet), via direct or indirect connection, or via some other network. Computational unit 103 may have significant ability to run multiple instances of the described method in parallel. Computational unit 103 may also have significant bandwidth for communication of data between multiple instances of described method. Users may interact with the computational unit 103 using remote devices 107, 109, e.g., using a web browser to connect to the computational unit 103 via one or more externally exposed web sites hosted by web server 105. Devices 107, 109 may be used in concert with computational unit 103 to access data stored therein, or may be used for other purposes. For example, from device 107 a user may access web server 105 using an Internet browser, as is known in the art, or by executing a software application that communicates with web server 105 and/or computational unit 103 over a computer network (such as the Internet).

Servers and applications may be combined on the same physical machines, and retain separate virtual or logical addresses, or may reside on separate physical machines. FIG. 1 illustrates just one example of a network architecture that may be used, and those of skill in the art will appreciate that the specific network architecture and data processing devices used may vary, and are secondary to the functionality that they provide, as further described herein. For example, services provided by web server 105 and computational unit 103 may be combined on a single server.

Each component 103, 105, 107, 109 may be any type of known computer, server, or data processing device. Computational unit 103, e.g., may include a processor 111 controlling overall operation of the computational unit 103. Computational unit 103 may further include RAM 113, ROM 115, network interface 117, input/output interfaces 119 (e.g., keyboard, mouse, display, printer, etc.), and memory 121. I/O 119 may include a variety of interface units and drives for reading, writing, displaying, and/or printing data or files. Memory 121 may further store operating system software 123 for controlling overall operation of the data processing device 103, control logic 125 for instructing computational unit 103 to perform aspects described herein, and other application software 127 providing secondary, support, and/or other functionality which may or may not be used in conjunction with aspects described herein. The control logic may also be referred to herein as the computational unit software 125. Functionality of the computational unit software may refer to operations or decisions made automatically based on rules coded into the control logic, made manually by a user providing input into the system, and/or a combination of automatic processing based on user input (e.g., queries, data updates, etc.).

Memory 121 may also store data used in performance of one or more aspects described herein, including a first database 129 and a second database 131. In some embodiments, the first database may include the second database (e.g., as a separate table, report, etc.). That is, the information can be stored in a single database, or separated into different logical, virtual, or physical databases, depending on system design. Devices 105, 107, 109 may have similar or different architecture as described with respect to device 103. Those of skill in the art will appreciate that the functionality of data processing device 103 (or device 105, 107, 109) as described herein may be spread across multiple data processing devices, for example, to distribute processing load across multiple computers, to segregate transactions based on geographic location, user access level, quality of service (QOS), etc.

One or more aspects may be embodied in computer-usable or readable data and/or computer-executable instructions, such as in one or more program modules, executed by one or more computers or other devices as described herein. Generally, program modules include routines, programs, objects, components, data structures, etc. that perform particular tasks or implement particular abstract data types when executed by a processor in a computer or other device. The modules may be written in a source code programming language that is subsequently compiled for execution, or may be written in a scripting language such as (but not limited to) HTML or XML. The computer executable instructions may be stored on a computer readable medium such as a hard disk, optical disk, removable storage media, solid state memory, RAM, etc. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like. Particular data structures may be used to more effectively implement one or more aspects described herein, and such data structures are contemplated within the scope of computer executable instructions and computer-usable data described herein.

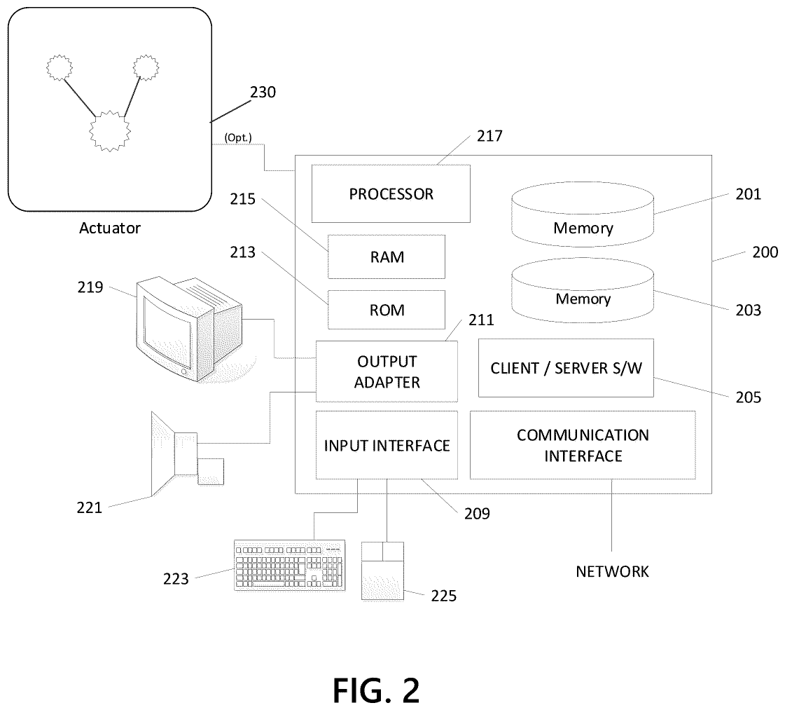

As described above, the computational unit 103 may perform methods described herein. FIG. 2 illustrates an example user device 200 such as device 107 (shown in FIG. 1) with which a user may access and communicate with computational unit 103. User device 200 and computational unit 103 may be part of the same device or may be separate devices. User device 200 may include a variety of components and modules including a processor 217, random access memory (RAM) 215, read only memory (ROM) 213, memories 201 and 203, which may include one or more collections of data, such as databases, client or server software 205, output adapter 211, input interface 209 and communication interface 207. Processor 217 may include a graphics processing unit (GPU) or a separate GPU may be included in the output adapter 211. Memory 201 may be configured to store electronic data, inclusive of any electronic information disclosed herein. Another memory, such as memory 203, may be configured to store different or overlapping data. In one embodiment, memories 201 and 203 may be a single, non-transitory computer-readable medium. Each memory 201, 203 may or may not include a database to store data or include data stored in RAM memory, accessed as needed by the client/server software. Data associated with the method described may be communicated between user device 200 and a computational unit 103 or a server through a transceiver or network interface, such as communication interface 207.

One or more statutory computer-readable mediums, such as medium 201 or 203 may be configured to contain sensor and/or server software (graphically shown as software 205). Sensor software 205 may, in one or more arrangements, be configured to identify domain-specific observational data as well as facilitate or direct communications between two devices, including remote devices 109 and/or communications devices, among other devices. A user may control the device, through input interface 209 using various types of input devices including keyboard 223 and mouse 225. Other types of input devices may include a microphone (e.g., for voice communications over the network), joysticks, motion sensing devices, touchscreens 219 and/or combinations thereof. In one or more arrangements, music or other audio such as speech may be included as part of the user experience with the device. Further collection of domain-specific observational data may be facilitated through cameras, GPS, accelerometers, chemical detectors, microscopes, or any other such input structures that may aid in gathering observational data. In such instances, the audio may be outputted through speaker 221. In some examples, this observational data may comprise domain-specific information. For example, the observational data may comprise biological information such as molecular-level information, atomic-level information, and/or other information corresponding to one or more biological phenomena.

In some embodiments, one or more actions suggested as query responses by the method described may be performed by actuators 230. These actuators 230 may comprise any structure or separate device which outputs directions to perform an action to a user or itself performs some of or all of the action dictated by the method described. Such actuators 230 may include, but are not limited to, various machines such as diagnostic devices, surgical devices, and/or other devices, appliances such as alarm clocks or washing machines, and robotic or artificially intelligent entities such as automated personal assistants. Such actuators 230 may be physically a part of user device 200 or computational unit (such as 103 shown in FIG. 1). Actuators 230 may also interact with user device 200 or computational unit 103 through a network (such as 101 shown in FIG. 1). Actuator 230 may provide query responses (e.g., instructions, recommended actions, or the like) pattern mappings (e.g., information and/or representations of information gathered by performing the methods described herein), and/or other outputs of methods described herein. Actuator 230 also may be used to perform specific tasks in order to solve a domain-specific (e.g., biological) problem which may be inputted into the method described. In a real-world setting, actuator 230 may perform any of a myriad of tasks including but not limited to providing a diagnostic test result, causing a chemical reaction, facilitating an experiment, and/or other functions.

Software 205, computer executable instructions, and other data used by processor 217 and other components of user device 200 may be stored in memories, 201, 203, RAM 215, ROM 213 or a combination thereof. Other types of memory may also be used, including both volatile and nonvolatile memory. Software 205 may be stored within RAM 215, ROM 213 and/or memories 201 and 203 to provide instructions to processor 217 such that when the instructions are executed, processor 217, device 200 and/or other components thereof are caused to perform functions and methods described herein. In one example, instructions for generating a user interface for interfacing with a server 105 or user device 107 may be stored in RAM 215, ROM 213 and/or databases 201 and 203. Software 205 may include both applications and operating system software, and may include code segments, instructions, applets, pre-compiled code, compiled code, computer programs, program modules, engines, program logic, and combinations thereof. Computer executable instructions and data may further be stored on some physical form of computer readable storage media (referred to herein as “computer memory”) including, e.g., electrically erasable programmable read-only memory (EEPROM), flash memory or other memory technology, CD-ROM, DVD or other optical disk storage, magnetic cassettes, magnetic tape, magnetic storage and the like. Software 205 may operate to accept and process observational data so that it may be used by the methods described. There may also be software 205 that relays actions dictated by the methods to the user. In some cases such software 205 may produce a text or voice list of instructions. Also, software 205 may allow an actuator to perform an action recommended by the method.

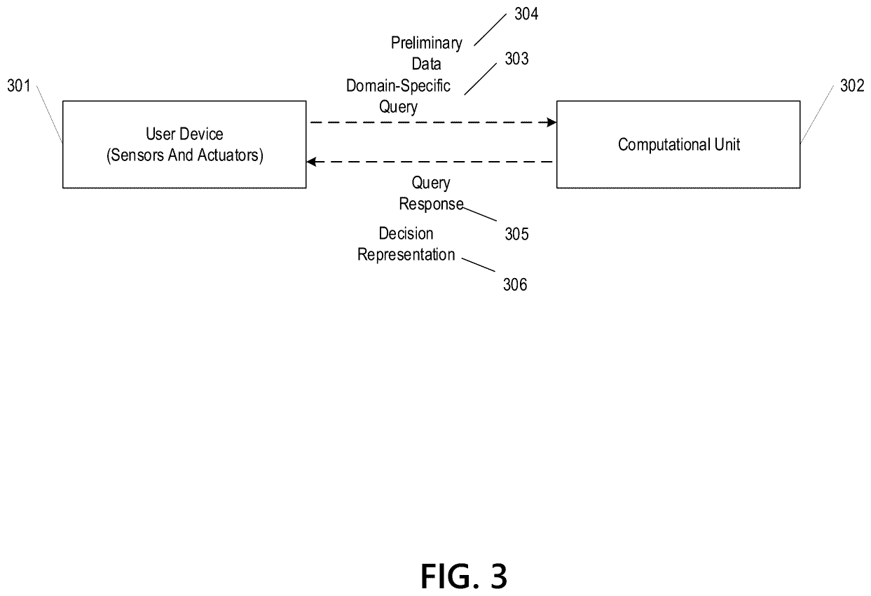

FIG. 3 shows a general view of possible communication between a user device 301 and the computational unit 302 which performs the methods described herein. In some examples, the computational unit 302 may be a component of and/or otherwise incorporated into the user device 301. In some examples, the computational unit 302 may be configured to provide one or more instructions (e.g., query responses) to the user device 301 to cause the user device 301 to perform one or more actions (e.g., via the actuators 230). In the shown embodiment, the user device 301 contains the input structures as well as the actuators. As described above, however, such a user device 301 need not contain all those elements. As shown in FIG. 3, the user device 301 may relay information to the computational unit 302. For example, the user device 301 may send, transmit, and/or otherwise relay information such as a domain-specific query (e.g., a request to solve a problem in the biological domain (e.g., provide a diagnosis, analyze test results, suggest a molecular compound for use in performing a task, and/or other any other biological problems), and/or a request to solve any other domain-specific query), preliminary data 304 (e.g., information captured by a sensor such as a camera, thermometer, barometer, potentiometer, microphone, and/or any other type of sensor, information provided by a computing device (e.g., a mobile phone, a laptop, a server, or the like) and/or other devices (e.g., microscopes, diagnostic devices, chemical detectors, and/or other devices), manually uploaded datasets (e.g., laboratory experiment results, preexisting datasets corresponding to biological problems, and/or other preliminary data), and/or other information), to the computational unit 302.

In some examples, a user may input a domain-specific query 303 into the user device to be relayed to the computational unit 302. Additionally and/or alternatively, in some examples, the domain-specific query 303 may be identified based on the preliminary data 304. The described preliminary data 304 and domain-specific query 303 may be communicated to the computational unit 302 through any of various methods of communication exemplified in the above description of FIG. 1. Using that the preliminary data 304 and/or the domain-specific query 303, the computational unit 302 performs the described explainable artificial intelligence methods in order to solve domain-specific problems while providing an explainable method for solving the problems. For example, the computational unit 302 may determine one or more steps to take, choices to make, contingent strategies to implement, and/or other actions to be performed by a robot and/or a human to respond to the domain-specific query 303. In some examples, the computational unit 302 may provide these responses to the user device 301 as a query response 305. In some examples, the computational unit 302 may perform these actions itself (e.g., by providing the query response to an actuator). In some examples, the computational unit 302 may determine the query response 305 based on providing the preliminary data 304 to a predictive artificial intelligence model trained using the methods described herein. The computational unit 302 may, by utilizing a predictive artificial intelligence model trained using the explainable artificial intelligence methods described herein, produce a decision representation 306 (e.g., a list, summary, pattern mapping, and/or other information corresponding to the query response 305 (e.g., data used by the model, correlations made, stored, and/or otherwise used by the model, decisions made by the model to produce a solution, and/or other information corresponding to the query response 305)) and/or other representations of steps taken by the predictive model to determine the query response 305.

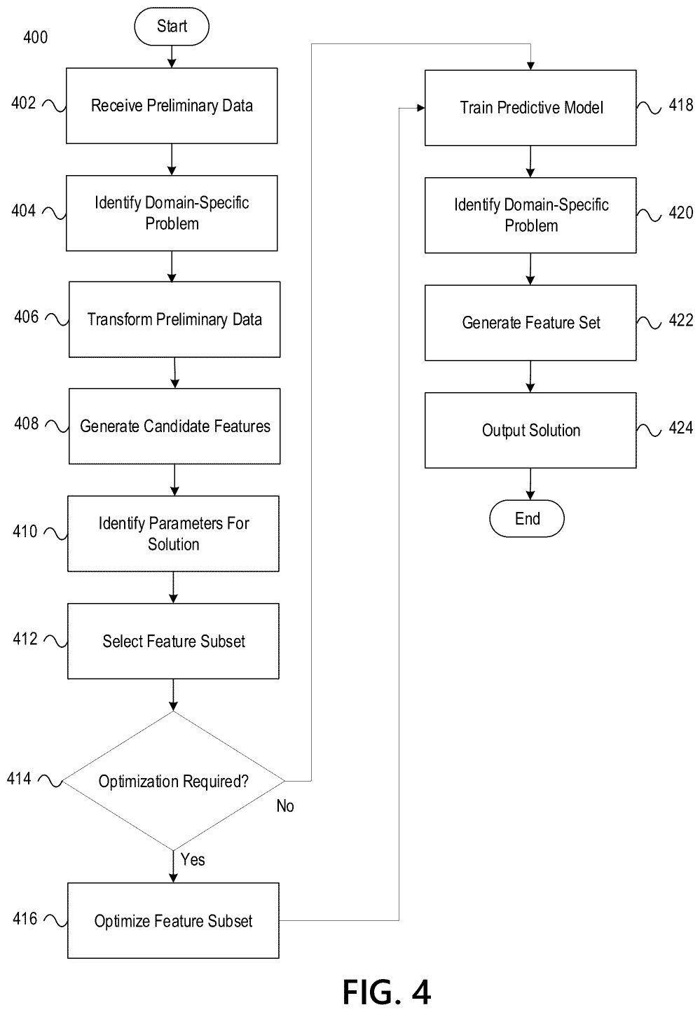

The computational unit 302 may perform explainable artificial intelligence methods for solving domain-specific problems as described herein. FIG. 4 provides an overview of steps of an explainable artificial intelligence method 400. Referring to FIG. 4, at step 402, an explainable artificial intelligence method may begin by receiving preliminary data. For example, the computational unit 302 may receive preliminary data corresponding to one or more domain-specific problem (e.g., a biological problem) from a user device 301. As described herein, the preliminary data may comprise information captured by a sensor such as a camera, thermometer, barometer, potentiometer, microphone, and/or any other type of sensor, information provided by a computing device (e.g., a mobile phone, a laptop, a server, or the like) and/or other devices (e.g., microscopes, diagnostic devices, chemical detectors, and/or other devices), domain-specific datasets (e.g., laboratory experiment results, preexisting datasets corresponding to biological problems, and/or other information relevant to solving a domain-specific problem. In some examples, the preliminary data may correspond to a real world environment (e.g., a geographic location, a laboratory setting, and/or other real world environments. In some examples, the preliminary data may correspond to a virtual or simulated world (e.g., a computer program, a virtual reality world, and/or other simulated worlds).

At step 404, the computational unit 302 may identify a domain-specific problem. In some examples, the computational unit 302 may identify the domain-specific problem based on the preliminary data. For example, the computational unit 302 may conduct a preliminary analysis of the preliminary data to identify a problem related to the preliminary data. In some examples, identifying the domain-specific problem may include identifying the optimal level of detail and/or description for the domain, as described herein. For example, the computational unit 302 may identify the optimal level of description based on balancing an estimated minimum of information required to solve the domain-specific problem and an estimated amount of computation time required to solve the domain-specific problem. The computational unit 302 may estimate the minimum information and/or the amount of computation time required based on historical information for related domain-specific problems, user inputs, and/or other parameters.

In the biology domain example, for instance, based on parsing, reading, and/or otherwise processing the preliminary data comprising an incomplete gene sequence, the computational unit 302 may identify a biological problem such completing the gene sequence. In some examples, the computational unit 302 may identify the domain-specific problem based on receiving a domain-specific query. For example, the computational unit 302 may receive, from the 301 (e.g., as part of the preliminary data of step 402 or separately from the preliminary data), a message, computer-executable instruction, and/or other query directing the computational unit 302 to solve a specific problem. For example, the computational unit 302 may receive, from the 301, a domain-specific query corresponding to the domain of biology, the domain of organic chemistry, and/or other domains.

At step 406, the computational unit 302 may transform preliminary data. In transforming the preliminary data, the computational unit 302 may automatically (e.g., without manual input) transform the preliminary data by converting, breaking down, and/or otherwise transforming preliminary data corresponding to one or more different levels of description (e.g., ecological, taxonomic, genomic, proteomic, molecular, atomic physics, and/or other levels of description) into a single uniform level of description. For example, the computational unit 302 may identify, based on the preliminary data, a plurality of categories of the preliminary data. Each category of the preliminary data may correspond to a level of description as described herein. Based on identifying the plurality of categories of the preliminary data, the computational unit 302 may reduce the preliminary data into a single category of information.

In order to provide benefits attributed to reducing the complexity of artificial intelligence forms required to solve domain-specific problems, as described herein, the computational unit 302 may transform preliminary data corresponding to a biological problem into molecular information. For example, based on preliminary data corresponding to an atomic physics level of description, the computational unit 302 may process (e.g., parse, cluster, categorize, and/or otherwise process) the preliminary data to identify one or more molecular interactions between, for example, molecules comprising atoms identified at the atomic physics level of description. Also or alternatively, the computational unit 302 may substitute similar atoms (e.g., in the same column of the periodic table) within molecular substructures involved in the molecular interactions). Accordingly, in these and other examples, the computational unit 302 may reformat, summarize, and/or otherwise transform the atomic physics information into molecular information. In some examples, in transforming the preliminary data, the computational unit 302 may additionally or alternatively convert preliminary data from a first unit of measurement to a second unit of measurement. For example, the computational unit 302 may convert one or more portions of the preliminary data into molar units as part of transforming the preliminary data into, for example, molecular information. In some examples, in transforming the preliminary data, the computational unit 302 may transform the preliminary data by indicating an operation or non-operation corresponding to a step of a biochemical pathway and/or by indicating one or more results of performing the step of the biochemical pathway.

At step 408, based on and/or by transforming the preliminary data, the computational unit 302 may generate a plurality of candidate features. The candidate features may comprise information corresponding to interactions between observable characteristics of a domain, corresponding to the preliminary data and/or to the domain-specific problem, and/or other subsets of information that may be used for training and/or utilizing a predictive AI model. For example, the candidate features may comprise information corresponding to interactions between objects, devices, organisms, molecules, environmental conditions, and/or other observable characteristics of a domain. In generating the plurality of candidate features, the computational unit 302 may perform one or more randomization operations on the transformed preliminary data and subsequently reduce, optimize, and/or otherwise revise the randomized candidate features into a set of candidate features corresponding to the domain-specific problem to be solved.

In some examples, the domain-specific problem may be a biological problem, as described herein. In these examples, in generating the plurality of candidate features as described above, the computational unit 302 may generate random combinations of molecular substructures corresponding to one or more molecules involved in the molecular interactions identified by the molecular information (e.g., the preliminary data transformed into molecular information), random combinations of aspects of molecules involved in a step of a biochemical pathway corresponding to a biological problem, random combinations of environmental conditions (e.g., temperature, pressure, or the like) corresponding to a step in a biochemical pathway, and/or random combinations of all of the above. In generating the random combinations, the computational unit 302 may use a combinatorial algorithm configured to combine aspects of molecules, environmental conditions, interactions between molecules, and/or other factors into information that is determinative of the operation of a step in at least one biochemical pathway related to the biological problem being solved. For example, the computational unit 302 may identify, based on the molecular interactions, a plurality of molecular substructures and subsequently generate randomized groupings of the molecular substructures. In some examples, the size of individual molecular substructures generated by the computational unit 302 may be based on one or more predetermined parameters included in the domain-specific query. In generating the candidate features, the computational unit 302 may generate a plurality of candidate molecular mechanisms based on the random combinations of molecular substructures. The candidate molecular mechanisms may comprise information indicating features involved in one or more steps of a biochemical pathway. For example, the candidate molecular mechanisms may comprise one or more aspects of molecules involved in a step of a biochemical pathway (e.g., molecular substructures, geometric relationships between molecules, molecular mass, molecular polarity, bonding properties, physical states, water solubility, melting temperature, boiling temperature, electrical conductivity, and/or other aspects of molecules). In some examples, the candidate molecular mechanisms may also or alternatively comprise combinations (e.g., combinations generated using a combinatorial algorithm, or the like) of molecular substructures corresponding to one or more actions to be taken (e.g., creating a vaccine, producing a chemical reaction, and/or other actions), combinations of molecular substructures corresponding to a downstream effect of the one or more actions to be taken (e.g., representations of molecular substructures included in an organism that will react to the one or more actions), representations of geometric relationships between the molecular substructures, catalysts/cofactors that contribute to a downstream effect of the one or more actions, and/or other information. For example, based on a biological problem of treating a disease, the computational unit 302 may generate candidate molecular mechanisms representative of the action of creating a treatment for the disease and the effect of the treatment on an organism carrying the disease. It should be understood that these examples are merely illustrative and that the computational unit 302 may generate additional or alternative molecular mechanisms (and/or other candidate features) based on any combinations of the transformed preliminary data without departing from the scope of this disclosure. Although a biological problem is described, it should be understood that additional or alternative problems in one or more different domains may also be solved using methods described herein. For example, a problem in the domain of organic chemistry may be solved, based on the steps of chemical synthesis cycles as opposed to the steps of biological pathways as described herein.

At step 410, the computational unit 302 may identify one or more parameters for a solution to the domain-specific problem. For example, the computational unit 302 may identify a set of selection parameters for determining a subset of candidate features to use in generating the solution to the domain-specific problem. In some examples, the set of selection parameters may comprise criteria for determining what subset of candidate features is relevant to the domain-specific problem. For example, if the problem is treatment of a disease, the set of selection parameters may comprise criteria indicating that candidate features corresponding to known treatments for the disease should be eliminated (e.g., to avoid producing redundant solutions to the biological problem). Additionally or alternatively, in some examples, the one or more parameters may comprise instructions to perform one or more optimization functions to optimize the features used to train and/or utilize a predictive AI model to produce a solution to the domain-specific problem. In some examples, in identifying the one or more parameters, the computational unit 302 may identify the one or more parameters within the domain-specific query. For example, the domain-specific query may have included the one or more parameters in addition to information identifying the domain-specific problem. In some examples, in identifying the one or more parameters, the computational unit 302 may output a prompt (e.g., to the user device 301, to a display associated with the computational unit 302, and/or to other devices) requesting user input of the one or more parameters.

In some examples, the computational unit 302 may identify one or more parameters indicating that the computational unit 302 should select a subset of the plurality of candidate features in order to optimize the features used to solve the domain-specific problem, conserve resources, and/or achieve other benefits. In these examples, the computational unit 302 may proceed to step 412. It should be understood that, in some examples, the computational unit 302 may use all of candidate features generated at step 408 to solve the biological problem. In these examples, the computational unit 302 may proceed to step 414 without performing the actions recited at step 412.

At step 412, the computational unit 302 may select an initial feature subset. The computational unit 302 may select the initial feature subset from the plurality of candidate features as part of a process of selecting an optimized (e.g., explainable) feature set comprising features that may be used, by a predictive AI model, to output the decisions, processes, steps, or the like used by the machine learning model to identify a solution to a domain-specific problem. For example, the computational unit 302 may select, from a plurality of candidate features comprising a plurality of candidate molecular mechanisms, a portion of the plurality of candidate molecular mechanisms. The computational unit 302 may select the initial feature subset based on the relevance of each of the selected candidate features to the overall domain-specific problem.

In some examples, the computational unit 302 may select the initial feature subset based on one or more downstream effects of individual candidate features. For example, if the domain-specific problem is a biological problem, the computational unit 302 may identify a plurality of biological effects that each correspond to at least one candidate molecular mechanism. In some examples, these biological effects may be the effects described with respect to step 408. For example, the biological effects may be and/or comprise effects of taking one or more actions involving the combinations of molecular substructures corresponding to a given candidate molecular mechanism. The biological effects may comprise the outcome of utilizing the combinations of molecular substructures (e.g., to diagnose an illness, prepare a treatment for a disease, cause a chemical reaction, and/or execute other uses for the combinations of molecular substructures).

In some examples, the computational unit 302 may select the initial feature subset by selecting the candidate features (e.g., molecular mechanisms) corresponding to the largest differential effects of performing one or more actions based on the interactions of the candidate features. For example, the computational unit 302 may identify a plurality of biological effects. Each biological effect may be and/or comprise a result of an interaction corresponding to at least one candidate molecular mechanism. In some examples, based on identifying the biological effects, the computational unit 302 may generate a plurality of effect scores. For example, the computational unit 302 may generate an effect score for each candidate molecular mechanism using one or more algorithms, ranking systems, or the like identified by the one or more parameters identified at step 410. The effect scores may indicate a degree and/or severity of a differential effect on the outcome, rate, or downstream effects of interactions corresponding to the candidate molecular mechanisms. In some examples, based on generating the effect scores, the computational unit 302 may compare each effect score to a threshold effect score. For example, the computational unit 302 may compare the effect scores to a threshold effect score identified by the one or more parameters in order to determine which effect scores satisfy (e.g., meet or exceed) the threshold effect score. In these examples, the computational unit 302 may select the initial feature subset by selecting a subset of molecular mechanisms, where each molecular mechanism of the subset corresponds to an effect score that satisfies the threshold effect score.

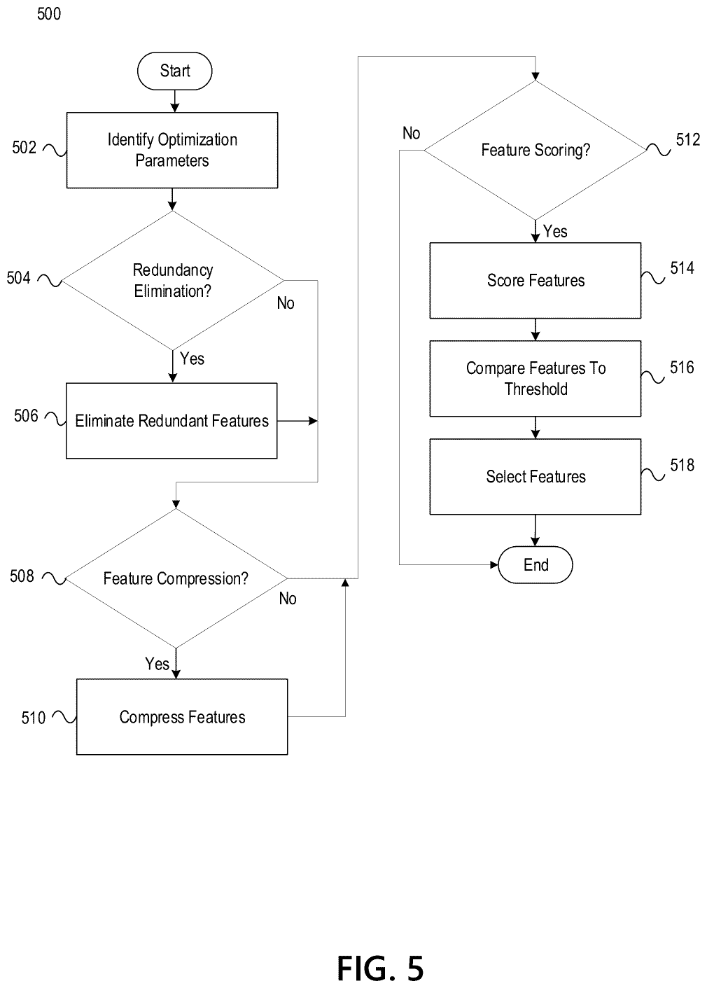

At step 414, based on selecting the initial feature subset, the computational unit 302 may identify whether optimization of the initial feature subset is required. For example, the computational unit 302 may identify, based on the domain-specific query and/or based on the one or more parameters, whether performance of one or more optimization actions to reduce the plurality of candidate features into an optimized feature subset is required. In some examples, the computational unit 302 may determine whether optimization of the initial feature subset is required as a binary yes/no choice. In some examples, in determining whether optimization of the initial feature subset is required, the computational unit 302 may identify a degree to which optimization is required. For example, the computational unit 302 may identify a number of optimization actions to be performed during optimization (e.g., redundancy elimination, feature compression, feature scoring, and/or other optimization actions as described herein, for example, with respect to FIG. 5). Based on identifying that optimization is required, the computational unit 302 may proceed to step 416 and optimize the feature subset. Based on identifying that optimization is not required, the computational unit 302 may proceed to step 418 without performing the functions recited at step 416.

At step 416, based on identifying that optimization of the initial feature subset is required, the computational unit 302 may optimize the initial feature subset. For example, the computational unit 302 may generate an optimized feature subset by performing one or more optimization actions to eliminate at least one candidate feature from the initial feature subset. In generating the optimized feature subset, the computational unit 302 may perform one or more of the optimization actions further described herein, for example, with respect to FIG. 5, and/or other optimization actions.

At step 418, the computational unit 302 may train a predictive AI model. For example the computational unit 302 may train the predictive AI model based on the optimized feature subset and/or based on the initial feature subset. In training the predictive AI model, the computational unit 302 may train a predictive AI model that comprises a simplified functional form relative to some conventional AI models. For example, rather than generating an AI model requiring a large training data set (e.g., a large language model, a deep neural network, or the like), the computational unit 302 may train a predictive AI model capable of making predictions with sharp transitions and/or discontinuities in the feature set used as training data without exceeding a predetermined amount of training data. The computational unit 302 may train, for example, a predictive AI model having the form of a shallow neural network. In these examples, final sigmoid activations may be used by the predictive AI model for classification tasks (e.g., transforming the preliminary data, and/or other classification tasks) and/or relu activations may be used for numerical prediction tasks (e.g., generating scores, and/or other prediction tasks). Also or alternatively, the computational unit 302 may train, for example, a predictive AI model having the form of one or more tree generation algorithms. For example, the predictive AI model may comprise algorithms for producing decision trees for classification tasks and/or producing regression trees for numerical prediction tasks. Also or alternatively, the computational unit 302 may train, for example, a predictive AI model comprising one or more ranking functions for predicting class rank differences based on individual features. It should be understood that, while the methods described indicate a preference for training a predictive AI model comprising a simplified functional form, the methods described herein may be applicable to other types of machine learning model.

In some examples, in training the predictive AI model, the computational unit 302 may train the predictive AI model based on the initial feature set and/or the optimized feature set as described herein. For example, the computational unit 302 may provide, as input, the initial feature set and/or the optimized features set as a training set for the predictive AI model. Training the predictive AI model may configure the predictive AI model to output query responses and/or representations of decisions made by the predictive model (e.g., pattern mappings, summaries, lists of steps, and/or other representations of decisions as described herein) for domain-specific queries. For example, training the predictive AI model may configure the predictive AI model to output, based on input of sets of molecular mechanisms, query responses and representations of decisions corresponding to solutions to biological problems. In some examples, to configure and/or otherwise train the predictive AI model, the computational unit 302 may cause the predictive AI model to process the initial feature set and/or the optimized feature set by applying natural language processing, natural language understanding, supervised machine learning techniques (e.g., regression, classification, neural networks, support vector machines, random forest models, naïve Bayesian models, and/or other supervised techniques), unsupervised machine learning techniques (e.g., principal component analysis, hierarchical clustering, K-means clustering, and/or other unsupervised techniques), and/or other techniques.

In some examples, in configuring and/or otherwise training the predictive AI model, the computational unit 302 may configure the predictive AI model to perform one or more of the functions described herein at steps 402-416. For example, the computational unit 302 may cause the predictive AI model to store one or more correlations between portions of the initial feature set and/or the optimized feature set and the functions recited at steps 402-416 used to produce the initial feature set and/or the optimized feature set, and/or otherwise cause the predictive AI model to perform one or more of the functions recited at steps 402-416 based on input of a domain-specific query, preliminary data, and/or sets of features (e.g., molecular mechanisms). It should be understood that, although steps 402-416 describe generating a single initial feature set and/or optimized feature set, in some examples the functions recited at steps 402-416 may be repeatedly performed to generate one or more additional feature sets prior to and/or after training the predictive AI model as described herein. In these examples, the additional feature sets may be used to update, refine, and/or otherwise further configure the predictive AI model output query responses and/or representations of decisions as described herein. Accordingly, the computational unit 302 may create an iterative loop configured to update, refine, and/or otherwise continuously or periodically configure the predictive AI model to improve the accuracy of query responses outputted by the predictive AI model and improve the efficiency of processes used to generate the query responses and representations of decisions (e.g., pattern mappings, or the like). For example, the computational unit 302 may configure the predictive AI model to perform an iterative update loop to output, based on performing one or more of the functions recited at steps 402-416, query responses and/or representations of decisions for additional domain-specific queries and refine the predictive AI model based on the results of these outputs. For example, the computational unit 302 may configure the predictive AI model to modify and/or otherwise update one or more algorithms used to generate query responses and/or representations of decisions as described herein based on identified patterns in domain-specific queries, user feedback to one or more query responses, and/or other results of outputting query responses and/or representations of decisions. In this way, the improvements over conventional methods described herein may be further enhanced by the iterative update loop.

At step 420, based on training the predictive AI model, the computational unit 302 may identify a domain-specific problem. For example, the computational unit 302 may identify an additional and/or new domain-specific problem based on further input from the user device 301. For example, the computational unit 302 may identify a domain-specific problem based on a domain-specific query requesting the predictive AI model be used to produce a query response. In these examples, the computational unit 302 may perform the functions described herein at steps 402-404 to identify the domain-specific problem and/or receive preliminary data corresponding to the domain-specific problem.

At step 422, the computational unit 302 may generate a feature set. For example, the computational unit 302 may generate an initial feature set and/or an optimized feature set corresponding to the domain-specific problem identified at step 420. In generating the feature set, the computational unit 302 may input, to the predictive AI model, preliminary data and/or a domain-specific query corresponding to the domain-specific problem. In these examples, the computational unit 302 may cause the predictive AI model to perform one or more of the functions recited at steps 406-416 to generate the feature set.

At step 424, based on generating the feature set, the computational unit 302 may output a solution. For example, the computational unit 302 may output a query response and/or a representation of decisions made by the predictive AI model to output the query response. In outputting the query response and/or the representation of decisions, the computational unit 302 may input the feature set of step 422 into the predictive AI model to cause the predictive AI model to output the query response and/or the representation of decisions. In outputting the query response, the predictive AI model may, for example, process the feature set. For example, the predictive AI model may identify one or more solutions to the biological problem based on comparing, combining, and/or otherwise identifying interactions between each feature in the feature set. For example, based on inputting a feature set comprising a plurality of molecular mechanisms, the computational unit 302 may cause the predictive AI model to generate simulations of some or all of set of possible effects of performing actions related to each molecular mechanism. Based on comparing, combining, and/or otherwise identifying the interactions between each feature in the feature set, the predictive AI model may identify which interactions between which features produce a solution to the domain-specific query corresponding to the feature set, as described further herein. In these examples, the predictive AI model may generate a query response comprising one or more instructions for solving the domain-specific query. For example, the computational unit 302 may cause the predictive AI model to output a query response comprising a formula, a genomic sequence, a series of treatments, and/or other methods of solving a biological problem.

In some examples, outputting the solution may comprise and/or cause application of one or more decisions made by the predictive AI model to one or more additional domain-specific problems. For example, outputting a solution comprising a representation of decisions made by the predictive AI model to output a solution to a domain-specific problem may cause one or more algorithms, machines, or human experts to utilize one or more of the decisions made by the predictive AI model to train an additional predictive AI model, output a solution for a problem adjacent to the domain-specific problem, and/or otherwise make use of the one or more decisions made by the predictive AI model to solve further domain-specific problems. For example, a solution for a problem adjacent to one or more biological problems solved by the predictive AI model may be generated and outputted based on defining a decision-making process for an additional predictive model and outputting, using the decision-making process, a solution for a biological problem comprising one or more parameters (e.g., environmental conditions, molecules involved, or the like) matching at least one parameter of the one or more biological problems for which the predictive AI model has previously outputted a solution. Also or alternatively, the one or more decisions may be used to revise the predictive AI model through an iterative loop. For example, the computational unit 302 may cause the predictive AI model to receive feedback information based on outputting the representation of decisions described above. The feedback information may comprise corrections to the query response, modified parameters, and/or other feedback from one or more machines and/or human experts in the domain corresponding to the domain-specific problem. In these examples, the iterative loop may comprise updating the predictive AI model based on the feedback information (e.g., by performing one or more configuration/training steps as described at step 420). The iterative loop may further comprise repeating the receiving feedback information and updating the predictive AI model for one or more biological problems until all biological problems for which a response is queried are solved.

In outputting the representation of decisions, the computational unit 302 may cause the predictive AI model to generate, while generating the query response, a pattern mapping, summary, list of steps, or the like comprising information and/or representations of information gathered by performing the methods described herein. For example, the predictive AI model may generate a “white box” summary of steps performing and/or interactions compared by the model in determining the query response. For example, the predictive AI model may generate a pattern mapping comprising information indicating one or more biological patterns identified by the model as the model compared molecular mechanisms to identify an optimal set of mechanisms for solving a biological problem.

In some examples, in outputting the query response and/or the representation of decisions, the computational unit 302 may send (e.g., transmit) the query response and/or the representation of decisions to the 301. In some examples, in sending the query response and/or the representation of decisions to the 301, the computational unit 302 may send one or more instructions causing the actuators to perform one or more functions to achieve the solution to the domain-specific (e.g., biological) problem.

It should be understood that steps 420-424 may be repeated each time the computational unit 302 receives a new domain-specific query in order to provide a query response and/or a representation of decisions using the predictive AI model. In these examples, the computational unit 302 may further refine and/or otherwise update the predictive AI model based on performing steps 420-424 for each new domain-specific query (e.g., as part of an iterative loop as described herein).

As described at step 416, the methods described herein may include optimizing feature sets (e.g., initial feature subsets). FIG. 5 illustrates steps of optimizing feature sets according to illustrative aspects described herein. Referring to FIG. 5, at step 502, the computational unit 302 may identify optimization parameters. For example, the computational unit 302 may perform, or have previously performed, the functions recited at step 410 and/or step 414 to identify one or more optimization actions to be performed (e.g., based on configuration settings, user instructions, or the like) as part of outputting a query response for a domain-specific query.

At step 504, the computational unit 302 may identify whether to perform redundancy elimination. For example, the computational unit 302 may identify whether the optimization parameters include instructions to eliminate, from the feature set, any redundant features (e.g., molecular mechanisms, and/or other features) that correspond to, for example, an indicator that a likelihood of the feature being a false positive exceeds a threshold likelihood (e.g., a predetermined score, value, or the like corresponding to a likelihood of a feature being a false positive). A false positive may be one of two or more candidate features having, for example, a similar predictive effect on a solution of the domain-specific problem, and/or other similarities. Based on identifying that redundancy elimination should be performed, the computational unit 302 may proceed to step 506 and eliminate redundant features. Based on identifying that redundancy elimination need not be performed, the computational unit 302 may proceed to step 508.

At step 506, the computational unit 302 may perform redundancy elimination. For example, the computational unit 302 may identify whether the feature set (e.g., the initial feature subset) comprises any redundant features. In identifying whether the features set comprises redundant features, the computational unit 302 may identify whether measurable differences between features meet or exceed a threshold tolerance level (e.g., a threshold difference in a number of observable features, a threshold difference in a number of unique features, a threshold similarity between the predictive effects of two or more features, and/or other tolerance levels). In some examples, the computational unit 302 may eliminate candidate features based on a comparative indicator (e.g., a score, a label, a ranking, or the like) of a likelihood of a candidate feature to be a false positive (e.g., a likelihood of the candidate feature having a similar predictive effect and/or value to solving the domain-specific problem). For example, the computational unit 302 may identify a plurality of biological effects. Each biological effect may correspond to at least one molecular mechanism included in the feature set (e.g., the initial feature subset). The computational unit 302 may compare each of these biological effects to identify, within a tolerance level, effects that are similar. For example, the computational unit 302 may compare two biological effects, such as two diagnostic effects corresponding to a molecular interaction, and identify that both effects produce the same downstream result in, for example, an organism. Also or alternatively, the computational unit 302 may identify, for example, that the molecular mechanisms corresponding to the two biological effects differ, in terms of composition, by only a single atom. Accordingly, the computational unit 302 may identify that the molecular mechanisms corresponding to the two biological effects meet or exceed a threshold tolerance level indicating a likelihood that the corresponding candidate features are false positives. The computational unit 302 may eliminate, from the feature set, redundant features. For instance, in the example of the two biological effects above, the computational unit 302 may eliminate one of the two features corresponding to the molecular mechanisms. In some examples, in performing redundancy elimination, the computational unit 302 may implement one or more additional or alternative comparison mechanisms to identify and eliminate redundant features.

In some examples, the computational unit 302 may eliminate candidate features based on the comparative indicator further indicating that instances of a molecular mechanism corresponding to a first candidate feature have been incorporated into instances of a second molecular mechanism corresponding to a different candidate feature. For example, based on a comparative indicator indicating that instances of the molecular mechanism corresponding to the first candidate feature are incorporated into instances of the second molecular mechanism exceed a frequency threshold (e.g., a limit, capacity, or the like regarding the number of times a molecular mechanism may be incorporated into instances of another molecular mechanism before being labeled as redundant), the computational unit 302 may eliminate the first candidate feature. Also or alternatively, in some examples, the elimination of the first candidate feature may be based on the size of the corresponding molecular mechanism. For example, if the molecular mechanism corresponding to the first candidate feature are is incorporated into instances of the second molecular mechanism in an amount exceeding the frequency threshold and the second molecular mechanism is larger, in size, than the molecular mechanism corresponding to the first candidate feature, the computational unit 302 may eliminate the first candidate feature.

At step 508, the computational unit 302 may identify whether to perform feature compression. For example, the computational unit 302 may identify whether the optimization parameters include instructions to compress features exceeding a threshold level of similarity. Based on identifying that feature compression should be performed, the computational unit 302 may proceed to step 510 and compress one or more features. Based on identifying that feature compression need not be performed, the computational unit 302 may proceed to step 512.

At step 510, the computational unit 302 may perform feature compression. Feature compression may comprise combining candidate features. In some examples, the computational unit 302 may combine candidate features by and/or based on combining a plurality of geometric constraints into a range of geometric constraints. In some examples, in performing feature compression, the computational unit 302 may combine one or more features (e.g., features corresponding to molecular mechanisms, or the like) based on their similarity. For example, the computational unit 302 may combine a plurality of molecular substructures sharing a threshold percentage (e.g., a threshold determined by a set of parameters provided as inputs, a threshold determined by a human expert, and/or other thresholds) of biochemical traits into a molecular substructure with a plurality of specification choices. The specification choices may comprise rules, permissions, or the like governing modifications that may be made to the molecular substructure to produce the solution to the domain-specific problem. For example, the specification choices may comprise permitting substitution of a first atom with a second atom in the same column of the periodic table, permitting substitution of a first catalyst with a second catalyst sharing a threshold percentage of similar traits (e.g., an effect on the rate of performing a reaction, and/or other traits). The computational unit 302 may determine the similarity of two or more given features based on comparing similarity scores for each feature. The similarity score may be and/or comprise values corresponding to the similarity in construction of two or more features (e.g., the information used to construct the features, the processes used to construct the features, or the like), the similarity in predictive effects of the features, and/or other similarities. In some examples, the computational unit 302 may compress features based on comparing the similarity scores to a threshold level of similarity and/or a threshold similarity score. For example, the computational unit 302 may compress two or more features (e.g., molecular mechanisms, or the like) that exceed a threshold level of similarity into a single feature. The threshold level of similarity may be or comprise a predetermined benchmark, score, test, or the like included in the domain-specific query and/or the one or more parameters.

In identifying whether a feature exceeds a threshold level of similarity, the computational unit 302 may compare one or more observable characteristics of features. For example, the computational unit 302 may identify a plurality of atoms corresponding to the feature set (e.g., the initial feature subset). For example, the computational unit 302 may identify which atoms correspond to each molecule and/or molecular substructure involved in a given molecular mechanism. Based on comparing the atoms for each molecule and/or molecular substructure, the computational unit 302 may identify that, for example, two molecular mechanisms are identical (e.g., in construction, function and/or effect) except for the substitution of similar atoms (e.g., atoms occupying the same column of the periodic table) in one molecular mechanism. In these examples, the computational unit 302 may compress the two molecular mechanisms into a single molecular mechanism. For example, the computational unit 302 may generate a new molecular mechanism comprising an indicator, for each atom corresponding to the molecular mechanism, of whether an atom may be substituted for a similar atom. By performing feature compression, the computational unit 302 may reduce or eliminate the presence of confounding variables (i.e., variables that might, due to their similarity in construction and/or predictive effects, cause a predictive AI model trained based on the variables to confuse their respective effects) in an optimized feature set.

At step 512, the computational unit 302 may identify whether to perform feature scoring. For example, the computational unit 302 may identify whether the optimization parameters include instructions to score features according to, for example, predictive power and eliminate features from the feature set based on the feature scoring. Based on identifying that feature scoring should be performed, the computational unit 302 may proceed to step 514 and score one or more features. Based on identifying that feature scoring need not be performed, the computational unit 302 may end performance of the method 500.

At step 514, the computational unit 302 may score one or more features. In some examples, the computational unit 302 may score features, based on one or more predetermined parameters, algorithms, or the like, to indicate the predictive power of a feature (i.e., the likelihood of the feature predicting a correct solution to the domain-specific query). For example, the computational unit 302 may, based on an algorithm included in the optimization parameters, generate a predictive score for each feature based on the complexity of the feature, the cost of performing one or more actions corresponding to the feature, and/or other variables indicative of the likelihood of the feature predicting a solution to the domain-specific problem. Also or alternatively, in some examples, the computational unit 302 may generate the predictive score for a feature based on whether the computational unit 302 previously performed redundancy elimination, feature compression, and/or any other optimization actions. For example, the computational unit 302 may increase the predictive score for a feature if the feature is a compressed feature, indicating an increased likelihood that the feature will be useful in predicting a solution to a domain-specific (e.g., biological) problem.

At step 516, the computational unit 302 may compare features to a threshold. For example, the computational unit 302 may compare a predictive score corresponding to each feature included in the feature set to a threshold predictive score. In comparing the predictive scores to the threshold predictive score, the computational unit 302 may determine whether each predictive score meets or exceeds the threshold predictive score.

At step 518, the computational unit 302 may select features from the feature set to select, retain, and/or otherwise produce a subset of candidate features. For example, the computational unit 302 may eliminate features from the feature set based on the results comparing the predictive scores for each feature to the threshold predictive score. For example, the computational unit 302 may eliminate any features corresponding to a predictive score that does not meet or exceed the threshold predictive score.

It should be understood that the optimization actions described herein are merely illustrative, and that the optimization actions described with respect to FIG. 5 may be performed in a different order without departing from the scope of this disclosure. For example, feature scoring and selection of a subset of candidate features may be performed first, elimination of candidate features may be performed second, and feature compression may be performed third. Further, it should be understood that one or more additional or alternative optimization actions may be performed to generate an optimized feature set without departing from the scope of this disclosure.

### III. Illustrative Use Case Scenario

The components of the described explainable artificial intelligence method will now be explained in an example of a computational unit analyzing a biomarker assay performed on a panel of human patients, as is described further herein in Part I. In this example, the computational unit may receive a domain-specific query requesting a diagnostic prediction for the different stages, variants, or physiologic expressions of a disease.

The device configuration may include a personal computer (e.g., bellowing to a physician, researcher, or the like) as the user device, and a remote server farm maintained by an entity (e.g., a hospital, a service provider, or the like) as the computational unit. These two may communicate over the wireless and wired Internet. To begin the process, the computational unit may receive the biomarker assay as preliminary data and the query from the user device.

The computational unit may then transform the biomarker assay (e.g., some measure of the amount of the biomarker present in each patient's case, as measured by means of some biochemical assay, such as, for example, various types of genomic sequencing, chromatography, or electrophoresis; and a diagnostic category assigned to that patient case) into a plurality of candidate molecular mechanisms using the functions described herein. For example, each biochemical reaction and/or binding interaction detailed in the biomarker assay may be analyzed to determine molecular substructures involved in the reaction or interaction. The computational unit may select an initial feature subset comprising a portion of the plurality of candidate molecular mechanisms. The computational unit may subsequently refine the initial feature subset into an optimized feature subset using one or more optimization actions as described herein. Based on generating the optimized feature subset, the computational unit may train one or more predictive AI models to output query responses and/or representation of decisions that may be used by the physicians and/or researchers to research, treat, or cure the disease corresponding to the biomarker assay. Subsequently, the computational unit may receive biomarker assay results for new sets of patients and use the one or more predictive AI models to produce query responses and/or representation of decisions that aid in understanding and/or solving the biological problem (i.e., treating the disease). In some examples, the representations of decisions may be used and/or otherwise applied to additional predictive models for solving additional (e.g., adjacent) domain-specific problems. Also or alternatively, in some examples, the computational unit may output, to the user device, instructions (e.g., audio instructions, written instructions, actuator commands, or the like) directing the user device and/or the user to solve the biological problem.

Hereinafter, various characteristics will be highlighted in a set of numbered clauses or paragraphs. These characteristics are not to be interpreted as being limiting on the invention or inventive concept, but are provided merely as a highlighting of some characteristics as described herein, without suggesting a particular order of importance or relevancy of such characteristics.

The following paragraphs (M1) through (M15) describe examples of methods that may be implemented in accordance with the present disclosure.
(M1) A method comprising: receiving, at a computing device, preliminary data corresponding to one or more biological problems; generating, based on a transformation of the preliminary data, a plurality of candidate features, each feature comprising a molecular mechanism, wherein a given molecular mechanism comprises: information that is determinative of the operation of a step in at least one biochemical pathway corresponding to the one or more biological problems, wherein the determinative information may comprise one or more of: one or more aspects of molecules involved in the step of the at least one biochemical pathway, or one or more environmental conditions corresponding to the step of the at least one biochemical pathway; reducing, by performing one or more feature reduction operations, the plurality of candidate features into an optimized feature set, wherein the one or more feature reduction operations comprise one or more of: selecting, based on one or more predictive scores indicating a likelihood of a given candidate feature to be predictive of a solution to a biological problem, a subset of candidate features, eliminating, based on a comparative indicator of the likelihood of a first candidate feature, of the plurality of candidate features, to be a false positive, the first candidate feature, wherein the comparative indicator indicates that the likelihood of the first candidate feature to be a false positive exceeds a threshold, or combining, based on one or more similarity scores, two or more candidate features with predictive effects on one or more solutions to the one or more biological problems and corresponding to matching similarity scores of the one or more similarity scores; training, based on the optimized feature set, a predictive model, wherein training the predictive model configures the predictive model to output solutions to biological problems and representations of decisions made by the predictive model to output the solutions to biological problems; outputting, using the predictive model, one or more solutions for the one or more biological problems; outputting, using the predictive model, a representation of decisions made by the predictive model to output the one or more solutions; and applying, based on the representation of decisions made by the predictive model to output the one or more solutions, one or more decisions made by the predictive model to at least one additional predictive model.
(M2) The method as described in paragraph (M1), wherein the applying the one or more decisions comprises: revising, through an iterative loop and based on the one or more solutions for the one or more biological problems, the predictive model, wherein the iterative loop comprises: receiving, based on outputting the representation of decisions made by the predictive model, feedback information corresponding to one or more human biology experts; updating, based on the feedback information, the predictive model; and repeating, during the outputting the one or more solutions for the one or more biological problems, the receiving feedback information and the updating until a solution has been outputted for each biological problem of the one or more biological problems.
(M3) The method as described in any of paragraphs (M1) through (M2), wherein the applying the one or more solutions one or more decisions made by the predictive model to the at least one additional predictive model comprises: defining, based on the one or more decisions made by the predictive model, a decision-making process for the at least one additional predictive model; and outputting, using the at least one additional predictive model, a solution for a problem adjacent to the one or more biological problems, wherein the problem adjacent to the one or more biological problems comprises one or more parameters matching at least one parameter of the one or more biological problems.
(M4) The method as described in any of paragraphs (M1) through (M3), wherein the given molecular mechanism further comprises one or more of: molecular substructures involved in the step of the at least one biochemical pathway, geometric constraints between one or more molecular substructures corresponding to a single molecule of a plurality of molecules involved in the step of the at least one biochemical pathway, catalysts affecting a rate of the step of the at least one biochemical pathway, or environmental conditions affecting the step of the at least one biochemical pathway.
(M5) The method as described in any of paragraphs (M1) through (M4), wherein the selecting of the subset of candidate features is based on one or more differential effects of each molecular mechanism, corresponding to the plurality of candidate features, on at least one outcome of solving the one or more biological problems.
(M6) The method as described in any of paragraphs (M1) through (M5), wherein the eliminating the first candidate feature is based on the comparative indicator further indicating that instances of a first molecular mechanism corresponding to the first candidate feature being incorporated into instances of a second molecular mechanism, corresponding to at least one second candidate feature and having a size exceeding the first molecular mechanism, exceed a frequency threshold.
(M7) The method as described in any of paragraphs (M1) through (M6), wherein the combining candidate features comprises one or more of: combining a plurality of geometric constraints into a range of geometric constraints, or combining a plurality of molecular substructures sharing a threshold percentage of biochemical traits into a molecular substructure with a plurality of specification choices, where the plurality of specification choices comprise one or more of: permitting substitution of a first atom with a second atom in the same column of the periodic table, or permitting substitution of a first catalyst with a second catalyst sharing a threshold percentage of similar traits.
(M8) The method as described in any of paragraphs (M1) through (M7), wherein the predictive model comprises: a ranking function, a decision tree, a random forest of decision trees, or a shallow neural network.
(M9) The method as described in any of paragraphs (M1) through (M8), wherein the transformation of the preliminary data comprises at least one of: an indication of an operation or non-operation corresponding to the step of the at least one biochemical pathway, or an indication of one or more results of performing the step of the at least one biochemical pathway.
(M10) A method for providing explainable artificial intelligence, comprising: receiving, at a computing device, a domain-specific dataset corresponding to a domain-specific problem; identifying, based on balancing an estimated minimum of information required to solve the domain-specific problem and an estimated amount of computation time required to solve the domain-specific problem, an optimal level of description for a domain corresponding to the domain-specific problem; generating, based on the domain-specific dataset, a plurality of candidate features, wherein generating the plurality of candidate features comprises: transforming one or more portions of the domain-specific dataset to correspond to the optimal level of description; and generating, based on the transforming and using a combinatorial algorithm, a plurality of combinations of features corresponding to the optimal level of description and included in the domain-specific dataset; reducing, by performing one or more feature reduction operations, the plurality of candidate features into an optimized feature set, wherein the one or more feature reduction operations comprise one or more of: selecting, based on based on one or more predictive scores indicating a likelihood of a given candidate feature predicting a solution to the domain-specific problem, a subset of candidate features, eliminating one or more redundant features, or compressing the plurality of candidate features to combine features exceeding a threshold similarity score; training, based on the optimized feature set, a predictive model, wherein training the predictive model configures the predictive model to output solutions to domain-specific problems and representations of decisions made by the predictive model to output the solutions to domain-specific problems; outputting, using the predictive model, a solution for the domain-specific problem and a representation of decisions made by the predictive model to output the solution; and applying, based on the representation of decisions made by the predictive model to output the solution, one or more decisions made by the predictive model to at least one additional domain-specific problem.
(M11) The method as described in paragraph (M10), wherein the applying the one or more decisions comprises: revising, through an iterative loop and based on the solution to the domain-specific problem, the predictive model, wherein the iterative loop comprises: receiving, based on outputting the representation of decisions made by the predictive model, feedback information corresponding to one or more human experts in the domain corresponding to the domain-specific problem; updating, based on the feedback information, the predictive model; and repeating, for one or more additional domain-specific problems, outputting a solution, the receiving feedback information, and the updating for each additional domain-specific problem.
(M12) The method as described in any of paragraphs (M10) through (M11), wherein the optimal level of description corresponds to descriptive information that has the following properties: the descriptive information indicates distinctions between candidate features corresponding to potential predictive values; a size of the descriptive information is below a threshold storage capacity; and the descriptive information comprises all information identified as necessary to predict a solution to the domain-specific problem.
(M13) The method as described in any of paragraphs (M10) through (M12), wherein the predictive model comprises: a ranking function, a decision tree, a random forest of decision trees, or a shallow neural network.
(M14) The method as described in any of paragraphs (M10) through (M13), wherein eliminating the one or more redundant features comprises eliminating, based on a comparative indicator of the likelihood of a first candidate feature, of the plurality of candidate features, to be a false positive exceeding a threshold, the first candidate feature.
(M15) The method as described in any of paragraphs (M10) through (M14), wherein the compressing comprises combining, based on comparing one or more similarity scores corresponding to respective features of the plurality of candidate features, two or more candidate features exceeding the threshold similarity score.

The following paragraphs (A1) through (A6) describe examples of computing systems that may be implemented in accordance with the present disclosure.
(A1) A computing system comprising: one or more processors; memory storing computer executable instructions that, when executed by the processor, cause the computing system to: receive a domain-specific dataset corresponding to a domain-specific problem; identify, based on balancing an estimated minimum of information required to solve the domain-specific problem and an estimated amount of computation time required to solve the domain-specific problem, an optimal level of description for a domain corresponding to the domain-specific problem; generate, based on the domain-specific dataset, a plurality of candidate features, wherein generating the plurality of candidate features comprises: transforming one or more portions of the domain-specific dataset to correspond to the optimal level of description; and generating, based on the transforming and using a combinatorial algorithm, a plurality of combinations of features corresponding to the optimal level of description and included in the domain-specific dataset; reduce, by performing one or more feature reduction operations, the plurality of candidate features into an optimized feature set, wherein the one or more feature reduction operations comprise one or more of: selecting, based on based on one or more predictive scores indicating a likelihood of a given candidate feature predicting a solution to the domain-specific problem, a subset of candidate features, eliminating one or more redundant features, or compressing the plurality of candidate features to combine features exceeding a threshold similarity score; train, based on the optimized feature set, a predictive model, wherein training the predictive model configures the predictive model to output solutions to domain-specific problems and representations of decisions made by the predictive model to output the solutions to domain-specific problems; output, using the predictive model, a solution for the domain-specific problem and a representation of decisions made by the predictive model to output the solution; and apply, based on the representation of decisions made by the predictive model to output the solution, one or more decisions made by the predictive model to at least one additional domain-specific problem.
(A2) The computing system as described in paragraph (A1), wherein the instructions, when executed, configure the computing system to apply the one or more decisions by: revising, through an iterative loop and based on the solution to the domain-specific problem, the predictive model, wherein the iterative loop comprises: receiving, based on outputting the representation of decisions made by the predictive model, feedback information corresponding to one or more human experts in the domain corresponding to the domain-specific problem; updating, based on the feedback information, the predictive model; and repeating, for one or more additional domain-specific problems, outputting a solution, the receiving feedback information, and the updating for each additional domain-specific problem.
(A3) The computing system as described in any of paragraphs (A1) through (A2), wherein the optimal level of description corresponds to descriptive information that has the following properties: the descriptive information indicates distinctions between candidate features corresponding to potential predictive values; a size of the descriptive information is below a threshold storage capacity; and the descriptive information comprises all information identified as necessary to predict a solution to the domain-specific problem.
(A4) The computing system as described in any of paragraphs (A1) through (A3), wherein the predictive model comprises: a ranking function, a decision tree, a random forest of decision trees, or a shallow neural network.
(A5) The computing system as described in any of paragraphs (A1) through (A4), wherein the instructions, when executed, configure the computing system to compress the plurality of candidate features by combining, based on comparing one or more similarity scores corresponding to respective features of the plurality of candidate features, two or more candidate features exceeding the threshold similarity score.
(A6) A computing system comprising: one or more processors; memory storing computer executable instructions that, when executed by the processor, cause the computing system to perform the method as described in any of paragraphs (M1) through (M15).

The following paragraph (S1) describes an examples of systems of devices that may be implemented in accordance with the present disclosure.
(S1) A system comprising: a computing device configured to perform the method as described in any of paragraphs (M1) through (M15); and a user device configured to send domain-specific queries and preliminary data.

The following paragraph (CRM1) describes examples of computer-readable media that may be implemented in accordance with the present disclosure.
(CRM1) One or more non-transitory computer-readable media storing instructions that, when executed by a computing system comprising at least one processor, a communication interface, and memory, cause the computing system to perform the method as described in any one of paragraphs (M1) through (M15).

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A method for providing explainable artificial intelligence, comprising:
receiving, at a computing device, preliminary data corresponding to one or more biological problems;
generating, based on a transformation of the preliminary data, a plurality of candidate features, each feature comprising a molecular mechanism, wherein a given molecular mechanism comprises:
information that is determinative of the operation of a step in at least one biochemical pathway corresponding to the one or more biological problems,
wherein the determinative information may comprise one or more of:
one or more aspects of molecules involved in the step of the at least one biochemical pathway, or
one or more environmental conditions corresponding to the step of the at least one biochemical pathway;
reducing, by performing one or more feature reduction operations, the plurality of candidate features into an optimized feature set, wherein the one or more feature reduction operations comprise one or more of:
selecting, based on one or more predictive scores indicating a likelihood of a given candidate feature to be predictive of a solution to a biological problem, a subset of candidate features,
eliminating, based on a comparative indicator of the likelihood of a first candidate feature, of the plurality of candidate features, to be a false positive, the first candidate feature, wherein the comparative indicator indicates that the likelihood of the first candidate feature to be a false positive exceeds a threshold, or
combining, based on one or more similarity scores, two or more candidate features with predictive effects on one or more solutions to the one or more biological problems and corresponding to matching similarity scores of the one or more similarity scores;
training, based on the optimized feature set, a predictive model, wherein training the predictive model configures the predictive model to output solutions to biological problems and representations of decisions made by the predictive model to output the solutions to biological problems;
outputting, using the predictive model, one or more solutions for the one or more biological problems;
outputting, using the predictive model, a representation of decisions made by the predictive model to output the one or more solutions; and
applying, based on the representation of decisions made by the predictive model to output the one or more solutions, one or more decisions made by the predictive model to at least one additional predictive model.

2. The method of claim 1, wherein the applying the one or more decisions comprises:
revising, through an iterative loop and based on the one or more solutions for the one or more biological problems, the predictive model, wherein the iterative loop comprises:
receiving, based on outputting the representation of decisions made by the predictive model, feedback information corresponding to one or more human biology experts;
updating, based on the feedback information, the predictive model; and
repeating, during the outputting the one or more solutions for the one or more biological problems, the receiving feedback information and the updating until a solution has been outputted for each biological problem of the one or more biological problems.

3. The method of claim 1, wherein the applying the one or more solutions one or more decisions made by the predictive model to the at least one additional predictive model comprises:
defining, based on the one or more decisions made by the predictive model, a decision-making process for the at least one additional predictive model; and
outputting, using the at least one additional predictive model, a solution for a problem adjacent to the one or more biological problems,
wherein the problem adjacent to the one or more biological problems comprises one or more parameters matching at least one parameter of the one or more biological problems.

4. The method of claim 1, wherein the given molecular mechanism further comprises one or more of:
molecular substructures involved in the step of the at least one biochemical pathway,
geometric constraints between one or more molecular substructures corresponding to a single molecule of a plurality of molecules involved in the step of the at least one biochemical pathway,
catalysts affecting a rate of the step of the at least one biochemical pathway, or
environmental conditions affecting the step of the at least one biochemical pathway.

5. The method of claim 1, wherein the selecting of the subset of candidate features is based on one or more differential effects of each molecular mechanism, corresponding to the plurality of candidate features, on at least one outcome of solving the one or more biological problems.

6. The method of claim 1, wherein the eliminating the first candidate feature is based on the comparative indicator further indicating that instances of a first molecular mechanism corresponding to the first candidate feature being incorporated into instances of a second molecular mechanism, corresponding to at least one second candidate feature and having a size exceeding the first molecular mechanism, exceed a frequency threshold.

7. The method of claim 1, wherein the combining candidate features comprises one or more of:
combining a plurality of geometric constraints into a range of geometric constraints, or
combining a plurality of molecular substructures sharing a threshold percentage of biochemical traits into a molecular substructure with a plurality of specification choices, where the plurality of specification choices comprise one or more of:
permitting substitution of a first atom with a second atom in the same column of the periodic table, or
permitting substitution of a first catalyst with a second catalyst sharing a threshold percentage of similar traits.

8. The method of claim 1, wherein the predictive model comprises:
a ranking function,
a decision tree,
a random forest of decision trees, or
a shallow neural network.

9. The method of claim 1, wherein the transformation of the preliminary data comprises at least one of:
an indication of an operation or non-operation corresponding to the step of the at least one biochemical pathway, or
an indication of one or more results of performing the step of the at least one biochemical pathway.

10. A method for providing explainable artificial intelligence, comprising:
receiving, at a computing device, a domain-specific dataset corresponding to a domain-specific problem;
identifying, based on balancing an estimated minimum of information required to solve the domain-specific problem and an estimated amount of computation time required to solve the domain-specific problem, an optimal level of description for a domain corresponding to the domain-specific problem;
generating, based on the domain-specific dataset, a plurality of candidate features,
wherein generating the plurality of candidate features comprises:
transforming one or more portions of the domain-specific dataset to correspond to the optimal level of description; and
generating, based on the transforming and using a combinatorial algorithm, a plurality of combinations of features corresponding to the optimal level of description and included in the domain-specific dataset;
reducing, by performing one or more feature reduction operations, the plurality of candidate features into an optimized feature set, wherein the one or more feature reduction operations comprise one or more of:
selecting, based on based on one or more predictive scores indicating a likelihood of a given candidate feature predicting a solution to the domain-specific problem, a subset of candidate features,
eliminating one or more redundant features, or
compressing the plurality of candidate features to combine features exceeding a threshold similarity score;
training, based on the optimized feature set, a predictive model, wherein training the predictive model configures the predictive model to output solutions to domain-specific problems and representations of decisions made by the predictive model to output the solutions to domain-specific problems;
outputting, using the predictive model, a solution for the domain-specific problem and a representation of decisions made by the predictive model to output the solution; and
applying, based on the representation of decisions made by the predictive model to output the solution, one or more decisions made by the predictive model to at least one additional domain-specific problem.

11. The method of claim 10, wherein the applying the one or more decisions comprises:
revising, through an iterative loop and based on the solution to the domain-specific problem, the predictive model, wherein the iterative loop comprises:
receiving, based on outputting the representation of decisions made by the predictive model, feedback information corresponding to one or more human experts in the domain corresponding to the domain-specific problem;
updating, based on the feedback information, the predictive model; and
repeating, for one or more additional domain-specific problems, outputting a solution, the receiving feedback information, and the updating for each additional domain-specific problem.

12. The method of claim 10, wherein the optimal level of description corresponds to descriptive information that has the following properties:
the descriptive information indicates distinctions between candidate features corresponding to potential predictive values;
a size of the descriptive information is below a threshold storage capacity; and
the descriptive information comprises all information identified as necessary to predict a solution to the domain-specific problem.

13. The method of claim 10, wherein the predictive model comprises:
a ranking function,
a decision tree,
a random forest of decision trees, or
a shallow neural network.

14. The method of claim 10, wherein eliminating the one or more redundant features comprises eliminating, based on a comparative indicator of the likelihood of a first candidate feature, of the plurality of candidate features, to be a false positive exceeding a threshold, the first candidate feature.

15. The method of claim 10, wherein the compressing comprises combining, based on comparing one or more similarity scores corresponding to respective features of the plurality of candidate features, two or more candidate features exceeding the threshold similarity score.

16. A computing system comprising:
one or more processors; and
memory storing computer executable instructions that, when executed by the one or more processors, cause the computing system to:
receive a domain-specific dataset corresponding to a domain-specific problem;
identify, based on balancing an estimated minimum of information required to solve the domain-specific problem and an estimated amount of computation time required to solve the domain-specific problem, an optimal level of description for a domain corresponding to the domain-specific problem;
generate, based on the domain-specific dataset, a plurality of candidate features,
wherein generating the plurality of candidate features comprises:
transforming one or more portions of the domain-specific dataset to correspond to the optimal level of description; and
generating, based on the transforming and using a combinatorial algorithm, a plurality of combinations of features corresponding to the optimal level of description and included in the domain-specific dataset;
reduce, by performing one or more feature reduction operations, the plurality of candidate features into an optimized feature set, wherein the one or more feature reduction operations comprise one or more of:
selecting, based on based on one or more predictive scores indicating a likelihood of a given candidate feature predicting a solution to the domain-specific problem, a subset of candidate features,
eliminating one or more redundant features, or
compressing the plurality of candidate features to combine features exceeding a threshold similarity score;
train, based on the optimized feature set, a predictive model, wherein training the predictive model configures the predictive model to output solutions to domain-specific problems and representations of decisions made by the predictive model to output the solutions to domain-specific problems;
output, using the predictive model, a solution for the domain-specific problem and a representation of decisions made by the predictive model to output the solution; and
apply, based on the representation of decisions made by the predictive model to output the solution, one or more decisions made by the predictive model to at least one additional domain-specific problem.

17. The computing system of claim 16, wherein the instructions, when executed, configure the computing system to apply the one or more decisions by:
revising, through an iterative loop and based on the solution to the domain-specific problem, the predictive model, wherein the iterative loop comprises:
receiving, based on outputting the representation of decisions made by the predictive model, feedback information corresponding to one or more human experts in the domain corresponding to the domain-specific problem;
updating, based on the feedback information, the predictive model; and
repeating, for one or more additional domain-specific problems, outputting a solution, the receiving feedback information, and the updating for each additional domain-specific problem.

18. The computing system of claim 16, wherein the optimal level of description corresponds to descriptive information that has the following properties:
the descriptive information indicates distinctions between candidate features corresponding to potential predictive values;
a size of the descriptive information is below a threshold storage capacity; and
the descriptive information comprises all information identified as necessary to predict a solution to the domain-specific problem.

19. The computing system of claim 16, wherein the predictive model comprises:
a ranking function,
a decision tree,
a random forest of decision trees, or
a shallow neural network.

20. The computing system of claim 16, wherein the instructions, when executed, configure the computing system to compress the plurality of candidate features by combining, based on comparing one or more similarity scores corresponding to respective features of the plurality of candidate features, two or more candidate features exceeding the threshold similarity score.
